(19) United States
(12) Patent Application Publication
(10) Pub. No.: US 2003/0170820 A1
(43) Pub. Date: Sep. 11, 2003

(54) Continuous in- vitro evolution

(76) Inventors: Gregory Coia, Brunswick (AU); Peter John Hudson, Blackburn (AU); Peter Iliades, North Balwyn (AU); Robert Alexander Irving, Mulgrave (AU)

Correspondence Address: FROMMER LAWRENCE & HAUG, 745 FIFTH AVENUE- 10TH FL., NEW YORK, NY, 10151, US

(21) Appl. No.: 10/408,930

(22) Filed: Apr. 7, 2003

Related U.S. Application Data
(63) Continuation-in-part of application No. 09/674,677, filed on Dec. 11, 2000, now Pat. No. 6,562,622.
Application No. 09/674,677 is a 371 of international application No. PCT/AU99/00341, filed on May 7, 1997.

(30) Foreign Application Priority Data
May 8, 1998 (AU) PP3445

(51) Int. Cl. C12P 21/02; C12N 5/06; C12N 15/86

(52) U.S. Cl. 435/69.1; 435/456; 435/320.1; 435/325

(57) ABSTRACT

Provided is a method for the mutation, synthesis and selection of a protein of interest, by first incubating a replicable RNA molecule encoding the protein with ribonucleoside triphosphate precursors of RNA and an RNA-directed RNA polymerase, such that the RNA-directed RNA polymerase replicates the RNA molecule but introduces mutations thereby generating a population of a mutant RNA molecules. The mutant RNA molecules are then incubated with a translation system under conditions which result in the synthesis of a population of mutant proteins. After translation, the mutant proteins are linked to their encoding RNA molecules, and one or more mutant proteins of interest are selected.

### RELATED APPLICATIONS

This application is a continuation-in-part of application Ser. No. 09/674,677, filed on Dec. 11, 2000, which is the National Phase of PCT/AU99/00341, filed May 7, 1999, designating the U.S. and published as WO 99/58661, with a claim of priority from Australian application no. PP 3445, filed May 8, 1998.

All of the foregoing applications, as well as all documents cited in the foregoing applications (“application documents”) and all documents cited or referenced in the application documents are incorporated herein by reference. Also, all documents cited in this application (“herein cited documents”) and all documents cited or referenced in herein cited documents are incorporated herein by reference. In addition, any manufacturer 's instructions or catalogues for any products cited or mentioned in each of the application documents or herein cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to a method for mutating and selecting novel proteins in a translation system; and to a polynucleotide construct for use in this method. The method of the present invention can be applied to the generation of molecules of diagnostic and therapeutic utility.

### BACKGROUND OF THE INVENTION

In vitro evolution of proteins involves introducing mutations into known gene sequences to produce a library of mutant sequences, translating the sequences to produce mutant proteins and then selecting mutant proteins with the desired properties. This process has the potential for generating proteins with improved diagnostic and therapeutic utilities. Unfortunately, however, the potential of this process has been limited by deficiencies in methods currently available for mutation and library generation.

For example, the generation of large libraries (e.g., beyond a library size of 10¹⁰) of unique individual genes and their encoded proteins has proven difficult with phage display systems, due to limitations in transformation efficiency. A further disadvantage is that methods which utilize phage-display systems (FIG. 1) require several sequential steps of mutation, amplification, selection and further mutation (Irving et al., 1996; Krebber et al., 1995; Stemmer, 1994; Winter et al., 1994).

Examples of procedures that have been used to date for affinity maturation of selected proteins, and particularly for the affinity maturation of antibodies, are set out in Table 1. All these methods rely on mutation of genes followed by display and selection of encoded proteins. The particular mutation method that is chosen determines the diversity in the resulting gene library. In vitro strategies (Table 1) are severely limited by the efficiency of transformation of mutated genes in forming a phage display library. In one in vivo cyclical procedure (Table 1, No.1), E. coli mutator cells were the vehicle for mutation of recombinant antibody genes. The E. coli mutator cells MUTD5-FIT (Irving et al., 1996), which bear a mutated DNAQ gene, could be used as the source of the S-30 extracts, and therefore allow mutations introduced into DNA during replication as a result of proofreading errors. However, mutation rates are low compared to the required rate. For example, to mutate 20 residues with the complete permutation of 20 amino acids requires a library size of 1×10²⁶, an extremely difficult task with currently available phage display methodology.

TABLE 1
Affinity maturation strategies

| | | Mechanism |
|---|---|---|
| | In vivo | |
| 1 | Mutator cells | Random point mutations |
| 2 | SIP-SAP | Co-selection and infection with antibody- |
| | | antigen pairs |
| | In vitro | |
| 3 | DNA shuffling-sexual | Recursive sequence recombination by DNA |
| | PCR | homology |
| 4 | Site directed | Oligonucleotide-coded mutations |
| | mutagenesis over | |
| | selected regions | |
| | (CDRs) | |
| 5 | Chain shuffling | Sequential replacement of heavy or light |
| | | chain domains using phage libraries |
| 6 | Error-prone PCR | Polymerase replication errors |

1) Irving et al. (1996); 2a) Krebber et al. (1995); 2b) Duenas and Borrebaeck (1994); 3) Stemmer (1994), Stemmer et al. (1995); 4) Yang et al. (1995); Sa) Barbas et al. (1994); 5b) Winter et al. (1994); 6) Gram et al (1992).

A selection method which enables the in vitro production of complex libraries of mutants which are continuously evolving (mutating) and from which a desired gene can be selected would therefore provide an improved means of affinity maturation (enhancement) of proteins.

In Vitro Coupled Transcription and Translation Systems

It is well known that a DNA plasmid containing a gene of interest can act as template for transcription when controlled by a control element such as the T7 promoter. It is also known that coupled cell-free systems may be used to simultaneously transcribe mRNA and translate the mRNA into peptides (Baranov et al 1993; Kudilicki et al. 1992; Kolosov et al 1992; Morozov et al 1993; Ryabova et al 1989, 1994; Spirin 1990; U.S. Pat. Nos. 5,556,769; 5,643,768; He and Taussig 1997). The source of cell free systems have generally been E. coli S-30 extracts (Mattheakis 1994; Zubay 1973) for prokaryotes and rabbit reticulocyte lysates for eukaryotes.

Transcription/translation coupled systems have also been reported (U.S. Pat. Nos. 5,492,817; 5,665,563; 5,324,637) involving prokaryotic cell free extracts (Mattheakis et al 1994) and eukaryotic cell free extracts (U.S. Pat. Nos. 5,492,817; 5,665,563) which have different requirements for effective transcription and translation. In addition, where selection of preferred mutant proteins is to occur directly from in vitro translated proteins there are separate requirements for the correct folding of the translated proteins in the prokaryotic and eukaryotic systems. For prokaryotes, protein disulphide isomerase (PDI) and chaperones may be required. Generally in prokaryotes translated proteins are folded after release from the ribosome; however, for correct folding of the newly translated protein attached (tethered) to the ribosome a C terminal anchor may also be necessary. An anchor is a polypeptide spacer that links the newly translated protein domain (s) to the ribosome. The anchor can be a complete protein domain such as an immunoglobulin constant region. In complete contrast to this, in eukaryotic systems the protein is folded as it is synthesized and has no requirement for the addition of prokaryote PDI and chaperones. An anchor can however be beneficial in eukaryotic systems for spacing of the newly translated protein from the ribosome and to facilitate correct folding as it remains attached (tethered) to the ribosome.

Polypeptides synthesized de novo in cell-free coupled systems have been displayed on the surface of ribosomes, since for example in the absence of a stop codon the polypeptide is not released from the ribosome. The mRNA ribosome protein complex can be used for selection purposes. This system mimics the process of phage display and selection and is shown in FIG. 1. Features required for optimal display on ribosomes have been described by Hanes and Pluckthun (1997). These features include removal of stop codons. However, removal of stop codons results in the addition of protease sensitive sites to the C terminus of the newly translated protein encoded by a ssrA tRNA-like structure. This can be prevented by the inclusion of antisense ssrA oligonucleotides (Keiler et al 1996).

RNA-Directed RNA Polymerases

Qβ bacteriophage is an RNA phage that infects E. coli. It has an efficient replicase (RNA-dependent RNA polymerases are termed replicases or synthetases) for replicating its single-strand RNA genome of coliphage Qβ. Qβ replicase is error-prone and introduces mutations into the RNA calculated in vivo to occur at a rate of one mutation in every 10³-10⁴ bases. The fidelity of Qβ replicase is low and strongly biased to replicating its template (Rohde et al 1995). These teachings indicate that replication over a prolonged period leads to accumulation of mutated strands not suitable for synthesis of a desired protein. Both + and − strands serve as templates for replicase; however, for the viral genome the + strand is bound by Qβ replicase and used as the template for the complementary strand (−). In order for RNA replication to occur the replicase requires specific RNA sequence/structural elements which have been well defined (Brown and Gold 1995; Brown and Gold 1996). A reaction containing 0.14 femtograms of recombinant RNA has been reported to be amplified by Qβ replicase to 129 nanograms in 30 mins (Lizardi et al 1988).

RNA-directed RNA polymerases are known to replicate RNA exponentially on compatible templates. Compatible templates are RNA molecules with secondary structure such as that seen in MDV-1 RNA (Nishihara, T., et al 1983). In this regard, a vector has been described for constructing amplifiable mRNAs as it possesses the sequences and secondary structure (MDV-1 RNA) required for replication and is replicated in vitro in the same manner as Qβ genomic RNA. The MDV-1RNA sequence (a naturally occurring template for Qβ replicase) is one of a number of natural templates compatible with amplification of RNA by Qβ replicase (U.S. Pat. No. 4,786,600); it possesses tRNA-like structures at its terminus which are similar to structures that occur at the ends of most phage RNAs which increase the stability of embedded mRNA sequences. Linearization of the plasmid allows it to act as a template for the synthesis of further recombinant MDV-1 RNA (Lizardi et al 1988). Teachings in the art show that prolonged replication by Qβ replicase of a foreign gene requires that it be embedded as RNA within one of the naturally occurring templates for Qβ such as MDV-1RNA.

### SUMMARY OF THE INVENTION

The present inventors have now found that RNA directed RNA polymerases introduce mutations into synthesised RNA molecules during replication in such a manner as to create a library of evolving (mutated) RNA molecules. These mutated RNA molecules vary in size due to insertions and deletions as well as point mutations and can be translated in vitro such that the corresponding proteins are displayed, for example, on a ternary complex comprising ribosome, RNA, and RNA encoded de novo synthesized protein. The present inventors have also identified conditions in which a large proportion of proteins generated by the ribosome display process are in a correctly folded, functional form. Furthermore, the present inventors have identified conditions in which phage Qβ replicase can function in eukaryotic coupled transcription/translation systems to amplify RNA templates, incorporating mutations into these templates.

The RNA molecules in the preferred transcription/translation system of the present invention are preferably in a continuous cyclic process of replication/mutation/translation leading to a continuous in vitro evolution (CIVE) process.

This CIVE process provides a novel method for in vitro evolution of proteins which avoids the limitation of numbers, library size and the time consuming steps inherent in previous affinity maturation processes.

Accordingly, in a first aspect the present invention provides a method for the mutation, synthesis and selection of a protein which binds to a target molecule, the method comprising:

(a) incubating a replicable RNA molecule encoding the protein with ribonucleoside triphosphate precursors of RNA and an RNA-directed RNA polymerase, wherein the RNA-directed RNA polymerase replicates the RNA molecule but introduces mutations thereby generating a population of mutant RNA molecules;

(b) incubating the mutant RNA molecules from step (a) with a translation system under conditions which result in the synthesis of a population of mutant proteins such that after translation, mutant proteins are linked to their encoding RNA molecules thereby forming a population of mutant protein/RNA complexes;

(c) selecting one or more mutant protein/RNA complex(es) by exposing the population of mutant protein/RNA complexes from step (b) to the target molecule and recovering the mutant protein/RNA complex(es) bound thereto; and

(d) optionally releasing or recovering the RNA molecules from the complex(es).

In a second aspect the present invention provides a method for the mutation, synthesis and selection of a protein which binds to a target molecule which includes:

(a) incubating a replicable RNA molecule encoding the protein with ribonucleoside triphosphate precursors of RNA and an RNA-directed RNA polymerase, wherein the RNA-directed RNA polymerase replicates the RNA molecule but introduces mutations thereby generating a population of mutant RNA molecules;

(b) incubating the mutant RNA molecules from step (a) with a translation system under conditions which result in the synthesis of a population of mutant proteins such that after translation, mutant proteins are linked to their encoding RNA molecules thereby forming a population of mutant protein/RNA complexes;

(c) selecting one or more mutant protein/RNA complex(es) by exposing the population of mutant protein/RNA complexes from step (b) to the target molecule;

(d) repeating steps (a) to (c) one or more times, wherein the replicable RNA molecule used in step (a) is the RNA obtained from complex(es) selected in step (c);

(e) recovering mutant protein complexes bound to the target molecule(s); and

(f) optionally releasing or recovering the RNA molecules from the complex(es).

The RNA from step (d) can be recycled through steps (a) to (c) without purification or isolation from the translation system.

In one embodiment, the RNA from step (d) is recycled via step (a) while the RNA is attached to the complex(es) obtained in step (c). In another embodiment, the RNA is released from the complex(es) obtained in step (c) prior to recycling. The RNA can be released from the complexes by any suitable mechanism. The mechanism can include raising the temperature of the incubation, or changing the concentration of the compounds used to maintain the complexes intact.

In the context of the present invention, the RNA can be recycled through steps (a) to (c) by sequential, manual steps. In a preferred embodiment, however, steps (a), (b), (c) and (d) are carried out simultaneously in a single reaction vessel and the recycling occurs automatically within the vessel.

In another embodiment of the second aspect, the RNA from step (d) can be transcribed into cDNA. The resulting cDNA can be cloned into a vector suitable for expression of the encoded protein.

In a third aspect the present invention provides a method for the mutation, synthesis and selection of a protein of interest, the method comprising:

(a) incubating a replicable RNA molecule encoding the protein with ribonucleoside triphosphate precursors of RNA and an RNA-directed RNA polymerase, wherein the RNA-directed RNA polymerase replicates the RNA molecule but introduces mutations thereby generating a population of mutant RNA molecules;

(b) incubating the mutant RNA molecules with a translation system under conditions which result in the synthesis of a population of mutant proteins such that after translation, mutant proteins are linked to their encoding RNA molecules; and

(c) selecting one or more mutant proteins of interest.

In a fourth aspect the present invention provides a method for producing and selecting a mutant protein of interest, the method comprising:

(a) incubating a replicable RNA molecule encoding the protein with ribonucleoside triphosphate precursors of RNA and an RNA-directed RNA polymerase, wherein the RNA-directed RNA polymerase replicates the RNA molecule but introduces mutations thereby generating a population of mutant RNA molecules;

(b) translating the mutant RNA molecules in cells such that after translation, each mutant protein is associated with or located within the same cell as its encoding RNA molecule; and

(c) selecting a cell comprising a mutant protein of interest.

The term “linked to”, as used herein, is intended to refer to an association between the translated protein and its encoding RNA, where the association is maintained through the processes of translation and selection, such that the RNA encoding the selected protein can be recovered. The translated protein and its encoding RNA can be linked to one another via a number of suitable linkage complexes.

For example, the complex can be a mitochondrion or other cell organelle suitable for protein display. In one particular embodiment, the complexes used to link translated proteins to their encoding RNAs are intact ternary ribosome complexes. A ribosome complex preferably comprises at least one ribosome, at least one RNA molecule and at least one translated polypeptide. This complex allows “ribosome display” of the translated protein. Conditions which are suitable for maintaining ternary ribosome complexes intact following translation are known. For example, deletion or omission of the translation stop codon from the 3′ end of the coding sequence results in the maintenance of an intact ternary ribosome complex. Sparsomycin or similar compounds can be added to prevent dissociation of the ribosome complex. Maintaining specific concentrations of magnesium salts and lowering GTP levels may also contribute to maintenance of the intact ribosome complex.

In another embodiment, the linkage complex can be a whole cell, i.e. a translated protein may be linked to its encoding RNA by virtue of association with or location within the same cell. A translated protein maybe “associated with” the same cell as its encoding RNA by, for example, being expressed on the surface of that cell or by being secreted from that cell.

In a further embodiment, the linkage is facilitated through an RNA binding protein. In this embodiment, the encoding RNA comprises a sequence encoding the protein of interest, a sequence encoding an RNA binding protein, and a sequence that may be bound by the de novo translated RNA binding protein (e.g. an RNA binding motif or domain). An example of a suitable RNA binding protein is the coat protein of phage MS2 that forms a complex with a TR 19-nt RNA hairpin structure (replicase translational operator). See, for example, Helgstrand et al 2002, Nucleic Acids Research, 30:2678. Another example of an RNA binding protein is the VP1 protein of Infectious Bursal Disease Virus (IBDV). The VP1 protein of IBDV is encoded by an RNA sequence to which it will bind. Accordingly, if the encoding RNA includes a coding sequence for VP1, the translated VP1 protein will bind to its own RNA sequence and hold together the quaternary ribosome complex.

In still another embodiment, the translated protein is fused to its encoding RNA. mRNA-protein fusions are described in Roberts, 1999, Current Opinion in Chemical Biology, 3:268. A covalent linkage between mRNA and a translated protein may be formed, for example, by puromycin as decribed by Nemoto et al., 1997, FEBS Lett. 414:405 and Roberts and Szostak, 1997, PNAS 94:12297.

It will be appreciated by those skilled in the art that preferred embodiments of the present invention involve coupled replication-translation-selection in a recycling batch process, and preferably, in a continuous-flow process (see, for example, FIG. 4). Continuous-flow equipment and procedures for translation or transcription-translation are known in the art and can be adapted to the methods of this invention by changing the composition of materials or conditions such as temperature in the reactor. Several systems and their methods of operation are reviewed in Spirin, A. S. (1991), which is incorporated by reference herein. Additional pertinent publications include Spirin et al. (1988); Rattat et al. (1990); Baranov et al. (1989); Ryabova et al. (1989); and Kigawa et al. (1991), all of which are incorporated by reference herein.

By “translation system” is meant a mixture comprising ribosomes, soluble enzymes, transfer RNAs, and an energy regenerating system capable of synthesizing proteins encoded by exogenous RNA molecules.

In one embodiment, the translation system is a cell-free translation system. Translation according to this embodiment is not limited to any particular cell-free translation system. The system may be derived from a eukaryote, prokaryote or a combination thereof. A crude extract, a partially purified extract or a highly purified extract can be used. Synthetic components can be substituted for natural components. Numerous alternatives are available and are described in the literature. See, for example, Spirin (1990b), which is incorporated by reference herein. Cell free translation systems are also available commercially. In one embodiment of the present invention the cell-free translation system utilises an S-30 extract from Escherichia coli. In another embodiment, the cell-free translation system utilizes a reticulocyte lysate, preferably a rabbit reticulocyte lysate.

The translation system can also comprise compounds that enhance protein folding. To this end, the Applicants have identified conditions in which an increased proportion of proteins produced by the ribosome display process are generated in a folded, functional form. These conditions include the addition of reduced and/or oxidized glutathione to the translation system at a concentration of between 0.1 mM and 10 mM. Preferably, the translation system comprises oxidized glutathione at a concentration of between 2 mM to 5 mM. Even more preferably, the translation system comprises oxidized glutathione at a concentration of about 2 mM and reduced gluthatione at a concentration of between 0.5 mM and 5 mM.

In another embodiment of the present invention, the translation system consists of or comprises a cell or compartment within a cell. The cell can be derived from a eukaryote or prokaryote.

If the translation system comprises whole cells, any suitable method can be used to introduce the mutant RNA molecules into the cells. For example, the mutant RNA molecules from step (a) can be introduced directly into the cells by any suitable transformation method. Alternatively, the mutant RNA molecules can be converted into DNA by reverse transcription prior to the transformation step. The resultant DNA molecules can be incorporated into replicable vectors in order to facilitate the transformation process. It will be understood that once the DNA molecules are introduced into the cells, mutant RNA molecules equivalent to those generated in step (a) are produced by transcription of the DNA molecules prior to translation.

In the context of the third and fourth aspects of the invention, any process of selecting a mutant protein of interest can be used. For example, selection can be achieved by binding to a target molecule or by measurement of a biological response affected by the mutant protein.

For example, if the protein of interest is an enzyme, the selection process can involve exposing mutant proteins to a target molecule, such as an enzyme substrate, and monitoring the enzymatic activity of the mutant proteins. The enzymatic activity can be monitored, for example, by analyzing whole cells or cell extracts comprising the mutant proteins.

In another example, if the protein of interest is an agent that promotes or reduces cell growth or division, the selection process can involve exposing mutant proteins to a population of cells and monitoring the biological responses of those cells.

In another example, if the mutant protein is a receptor ligand, the process can involve exposing mutant proteins to cells expressing the receptor and monitoring a biological response effected by signalling of the receptor.

A number of RNA-directed RNA polymerases (otherwise known as replicases or RNA synthetases) known in the art have been isolated and are suitable for use in the method of the present invention. Examples of these include bacteriophage RNA polymerases, plant virus RNA polymerases and animal virus RNA polymerases. In a preferred embodiment of the present invention, the RNA-directed RNA polymerase introduces mutations into the replicated RNA molecule at a relatively high frequency, preferably at a frequency of at least one mutation in 10⁴ bases, more preferably one mutation in 10³ bases. In a more preferred embodiment the RNA-directed RNA polymerase is selected from the group consisting of Qβ replicase, Hepatitis C RdRp, Vesicular Stomatitis Virus RdRp, Turnip yellow mosaic virus replicase (Deiman et al (1997) and RNA bacteriophage phi 6 RNA-dependent RNA polymerase (Ojala and Bamford (1995). Most preferably, the RNA-directed RNA polymerase is Qβ replicase.

The RNA-directed RNA polymerase can be included in the transcription/translation system as a purified protein. Alternatively, the RNA-directed RNA polymerase can be included in the form of a gene template which is expressed simultaneously with step (a), or simultaneously with steps (a), (b) and (c) of the methods of the first or second aspects of the present invention.

In a further preferred embodiment, the RNA-directed RNA polymerase can be fused with or associated with the target molecule. Without wishing to be bound by theory, it is envisaged that in some cases, the binding affinity of the translated protein for the target can be greater than the affinity of the replicase for the RNA molecule. The binding of the mutant protein/RNA complex to a target molecule/RNA-directed RNA polymerase fusion construct would bring the RNA into the proximity of the RNA-directed RNA polymerase. This may result in preferential further replication and mutation of RNA molecules of interest.

RNA templates that are replicated by various RNA-dependent RNA polymerases are known in the art and may serve as vectors for producing replicable RNAs suitable for use in the present invention. Known templates for Qβ replicase include RQ135 RNA, MDV-1 RNA, microvariant RNA, nanovariant RNAs, CT-RNA and RQ120 RNA. Qβ RNA, which is also replicated by Qβ replicase, is not preferred, because it has cistrons, and further because the products of those cistrons regulate protein synthesis. Preferred vectors include MDV-1 RNA and RQ135 RNA. The sequences of both are published. See Kramer et al. (1978) (MDV-1 RNA) and Munishkin et al. (1991) J (RQ135), both of which are incorporated by reference herein. They can be made in DNA form by well-known DNA synthesis techniques.

In a preferred embodiment of the first aspect of the present invention, the method further includes the step of transcribing a DNA construct to produce replicable RNA. DNA encoding the recombinant RNA can be, but need not be, in the form of a plasmid. It is preferable to use a plasmid and an endonuclease that cleaves the plasmid at or near the end of the sequence that encodes the replicable RNA in which the gene sequence is embedded. Linearization can be performed separately or can be coupled with transcription-replication-translation. Preferably, however, linear DNA is generated by any one of the many available DNA replication reactions and most preferably by the technique of Polymerase Chain Reaction (PCR). For some systems non-linearized plasmids without endonuclease may be preferred. Suitable plasmids can be prepared, for example, by following the teachings of Melton et al (1984a,b) regarding processes for generating RNA by transcription in vitro of recombinant plasmids by bacteriophage RNA polymerases, such as T7 RNA polymerase or SP6 RNA polymerase. See, for example, Melton et al. (1984a) and Melton (1984b), which are incorporated by reference herein. It is preferred that transcription begin with the first nucleotide of the sequence encoding the replicable RNA.

In a further preferred embodiment the transcription is carried out simultaneously in a single or multiple chambered reaction vessel, or reactor, with steps (a), (b), (c) of the method according to the first or second aspects of the present invention.

The target molecule used to select the mutant protein can be any compound of interest (or a portion thereof) such as a DNA molecule, a protein, an enzyme substrate, a receptor, a cell surface molecule, a metabolite, an antibody, a hormone, a bacterium, a virus, a small molecule, a carbohydrate or a lipid.

In a preferred embodiment, the target molecule is bound to a matrix and added to the reaction mixture comprising the complex (displaying translated proteins). The target molecule can be coated, for example, on a matrix such as magnetic beads. The magnetic beads can be Dynabeads®. It will be appreciated that the translated proteins will competitively bind to the target molecule. Proteins with higher affinity will preferably displace lower affinity molecules. Thus, the method of the present invention allows selection of mutant proteins that exhibit improved binding affinities for a target molecule of interest.

The Applicants have also made the surprising finding that minimal sequences derived from naturally occurring replicase templates, such as the MDV-1 template, are sufficient for the binding of Qβ replicase. On the basis of this finding a novel construct suitable for transcription of replicable RNA has been developed.

Accordingly, in a preferred embodiment of the first to the fourth aspects of the present invention, the method further includes transcribing a DNA construct to produce a replicable RNA molecule, wherein the DNA construct comprises:

(i) an untranslated region comprising a control element which promotes transcription of the DNA into RNA and a ribosome binding site;

(ii) an open reading frame encoding the protein which binds to the target molecule; and

(iii) a stem-loop structure situated upstream of the open reading frame.

In a fifth aspect the present invention provides a DNA construct comprising:

(i) an untranslated region comprising a control element which promotes transcription of the DNA into RNA and a ribosome binding site;

(ii) a cloning site located downstream of the untranslated region; and

(iii) a replicase binding sequence located upstream of the cloning site.

When used herein the phrase “replicase binding sequence” refers to a polynucleotide sequence which acts as a “loop-like” secondary structure which is recognized by a replicase (in particular, a replicase holoenzyme). Preferably, the replicase binding sequence does not include a full length RNA template for a replicase molecule. For example, preferably the phrase “replicase binding sequence” does not include full length MDV-1 RNA or RQ135 RNA templates.

In a preferred embodiment, the replicase binding sequence is between 15 to 50 nucleotides in length, more preferably between 20 and 40 nucleotides in length. Preferably, the replicase binding sequence is recognized by Qβ replicase.

In a further preferred embodiment, the sequence of the replicase binding sequence comprises or consists of the sequence: GGGACACGAAAGCCCCAGGAACCUUUCG (SEQ ID NO:25).

In a further preferred embodiment, a second replicase binding sequence is included downstream of the cloning site.

Any suitable ribosome binding site can be used in the construct of the present invention. Prokaryotic and eukaryotic ribosome binding sequences can be incorporated depending on whether prokaryotic or eukaryotic systems are being used. A preferred prokaryotic ribosome binding site is that of the MS2 virus.

In a further preferred embodiment, the DNA construct includes a translation initiation sequence. Preferably, the translation initiation sequence is ATG.

It will be apparent to those skilled in the art that any gene of interest can be inserted into the cloning site in the DNA construct. In a preferred embodiment, the gene(s) of interest is a nucleotide sequence coding for (i) a library of target binding proteins or (ii) a single target binding protein, where the target can include any of protein, DNA, cell surface molecules, receptors, antibodies, hormones, viruses or other molecules or complexes or derivatives thereof.

A nucleotide sequence coding for an anchor domain can be fused 3′ in frame with the gene of interest. The anchor domain can be any polypeptide sequence which is long enough to space the protein translated from the gene of interest a sufficient distance from the ribosome to allow correct folding of the molecule and accessibility to its cognate binding partner. Preferably, the polypeptide has a corresponding RNA secondary structure that mimics that of a replicase template. In a preferred embodiment, the polypeptide is an immunoglobulin constant domain. Preferably, the polypeptide is a constant light domain. The constant light domain can be the first constant light region of the mouse antibody 1C3. Preferably, the constant domain is encoded by the sequence shown in FIG. 5a. Alternatively, the polypeptide can be the human IgM constant domain. In another embodiment the anchor can be selected from the group consisting of: the octapeptide “FLAG” epitope, DYKDDDDK (SEQ ID NO:27) or a polyhistidine₆ tag followed optionally by a translation termination (stop) nucleotide sequence. The translation termination (stop) nucleotide sequence can be TAA or TAG. In some constructs of the present invention, no stop codons are present so as to prevent recognition by release factors and subsequent protein release. In these constructs, the anti-sense ssrA oligonucleotide sequence can be added to prevent addition of a C terminal protease site in the 3′ untranslated region that follows.

In a sixth aspect the present invention provides a kit for generating a replicable RNA transcript which includes a DNA construct according to the second aspect of the present invention.

In a preferred embodiment the kit includes at least one other additional component selected from

(i) an RNA-directed RNA polymerase, preferably Qβ replicase, or a DNA or RNA template encoding an RNA-directed RNA polymerase;

(ii) a cell free translation system;

(iii) a DNA directed RNA polymerase, preferably a bacteriophage;

(iv) ribonucleoside triphosphates; and

(v) restriction enzymes.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

### BRIEF DESCRIPTION OF THE DRAWINGS

The following Detailed Description, given by way of example, but not intended to limit the invention to specific embodiments described, may be understood in conjunction with the accompanying drawings, incorporated herein by reference, in which:

FIG. 1 shows the affinity maturation cycle for a) phage display and b) ribosome display in the continuous in-vitro evolution (CIVE) process.

FIG. 2 shows a schematic representation of an expression unit containing a gene of interest (nucleotide sequence) for CIVE. The expression unit comprises a gene of interest with upstream ribosome binding site (RBS) and translational initiation site (ATG) along with a transcriptional initiation sequence (T7 promoter). The construct also comprises a downstream spacer sequence.

FIG. 3 shows a schematic representation of the CIVE method showing the continuous cycling nature of in vitro affinity maturation. The method enables the in vitro production of complex libraries of mutants which are continuously evolving (mutating) and from which a desired gene can be selected; the RNA molecules in the preferred transcription/translation system of the present invention are in a continuous cyclic process of replication/mutation/translation leading to continuous in vitro evolution (CIVE).

FIG. 4 shows a representation of a reaction vessel suitable for the CIVE process.

FIG. 5 shows nucleotide sequences of: a) the first constant light region of mouse monoclonal antibody 1C3 (SEQ ID NO: 1); b) the third constant heavy region of the human IgM antibody (SEQ ID NO:2); c) the anti glycophorin (1C3) scFv (SEQ ID NO:3); d) the anti-HepatitisB surface antigen (4C2) scFv (SEQ ID NO:4).

FIG. 6 shows the DNA sequence of the plasmid pBRT7Qβ containing a cDNA copy of the Qβ bacteriophage genome (SEQ ID NO:5).

FIG. 7 shows a schematic representation of the plasmids (a) pGC038CL (containing the anti-glycophorin scFv (1C3) and the mouse constant light region) and (b) pGC_CH (containing the human constant heavy region), which were used for the PCR synthesis of template used for in vitro transcription and translation. These plasmids were used to supply downstream spacer sequences. In most cases, genes of interest were cloned into SfiI and NotI sites of pGC_CH.

FIG. 8 shows sequences of RNA fragments that form stem loop structures (SEQ ID NO:35 and SEQ ID NO:36).

FIG. 9 shows eukaryotic expression vector pcDNA3.1 for expression of Qβ replicase or Hepatitis C virus RNA dependent RNA polymerase in the rabbit reticulocyte coupled transcription/translation system.

FIG. 10 shows the DNA sequence of the Hepatitis C virus RNA dependent RNA polymerase (SEQ ID NO:6).

FIG. 11 shows DNA sequences of oligonucleotides used as primers in PCR reactions to generate template DNA for in vitro coupled transcription/translation reactions. Nucleotide sequences of oligonucleotides used for both the generation of templates and the recovery of products after panning. Sequences are numbered and are written 5′ to 3′ (SEQ ID NOs:7-24).

FIG. 12 shows expression of the Qβ replicase in the rabbit reticulocyte coupled transcription/translation system.

FIG. 13 shows the effect of Qβ replicase on coupled transcription/translation of anti GlyA 1C3 protein synthesis.

FIG. 14 shows the effect of including Qβ replicase in coupled transcription and translation; Table of mutations in the sequences of selected mutants. This figure shows the positions and type of mutations found in 280 nucleotides of sequence from 6 random clones. These had been recovered from pannings of the anti-GlyA scFv against GlyA coated Dynabeads® after transcription and translation either in the absence of Qβ replicase, in the presence of purified Qβ or in the presence of plasmid pcDNAQβ. In the “Mutation Found” column, “None” means that no mutations were found. Mutations are shown in the form AxB where A is the wild type nucleotide, x is the position number within the sequence (as presented in FIG. 5c) and B is the mutated nucleotide observed.

FIG. 15 shows replication of anti glycophorin scFv transcripts by Qβ replicase in the coupled transcription/translation rabbit reticulocyte system: densitometer scanning.

FIG. 16 shows DNA sequence analysis of replication and mutation of anti glycophorin scFv and anti Hepatitis B scFv by Qβ replicase from T7 polymerase transcripts.

FIG. 17 shows a vector containing the Hepatitis C RNA dependent RNA polymerase.

FIG. 18 shows the effect of Hepatitis C RNA dependent RNA polymerase expressed in the coupled transcription/translation system on replication of anti GlyA 1C3 scFv RNA. Agarose gel electrophoresis of the RT-PCR products stained with ethidium bromide and scanned.

FIG. 19 shows a schematic representation of the CIVE method used to produce mutant β-lactamase conferring increased resistance to cefotaxime on bacteria expressing the mutant gene.

FIG. 20 is a bar graph showing increased resistance to cefotaxime of bacteria expressing mutant β-lactamase genes obtained by the CIVE method of the present invention.

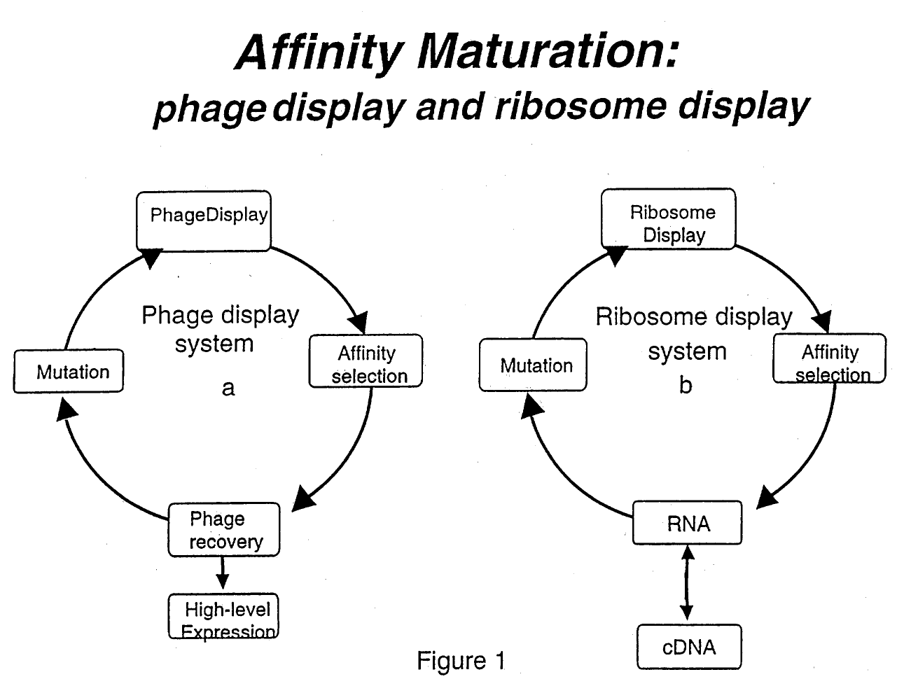

### DETAILED DESCRIPTION OF THE INVENTION

In a preferred aspect of the present invention, the system for continuous one-step evolution of proteins comprises the following components.

The Expression Unit

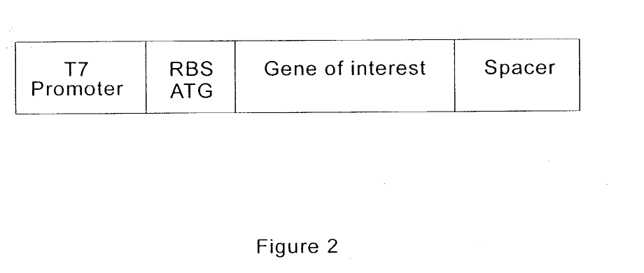

A preferred expression unit for use in the present invention is depicted in FIG. 2. This expression unit comprises 3′ and 5′ untranslated regions with in the 5′ untranslated region and a control element such as the T7 or SP6 promoter to promote transcription of the DNA into mRNA. The consensus DNA sequences are specific for their polymerases; the T7 promoter sequence for T7 RNA polymerase is:

TAATACGACTCACTATAGGGAGA (SEQ ID NO:26).

The T7 promoter sequence may act as an RNA dependent RNA polymerase binding sequence (i.e., it may act as a binding sequence for Qβ replicase). Preferably, however, the construct includes a stemloop structure for the binding of Qβ replicase, located in the 5′ untranslated region 3′ to the promoter site. Preferably, a second stemloop structure is included downstream of the coding sequence, preferably about 1 kb 3′ of the translation termination site of the expression unit. The preferred sequence of the stemloop structure is:

GGGACACGAAAGCCCCAGGAACCUUUCG (SEQ ID NO:25).

The ribosome binding site is the next region downstream of the promoter. Any of several ribosome binding sites can be used in this position. Prokaryotic and eukaryotic ribosome binding sequences may be incorporated depending on whether a eukaryotic or prokaryotic coupled system is being used. One preferred prokaryotic binding site is that of the MS2 virus. The translation initiation sequence ATG is preferably used and codes for the amino acid methionine; this is the start point for protein translation.

The Gene (Nucleotide Sequence) of Interest

It will be apparent to those skilled in the art that the gene of interest can be attached to the untranslated regions by any of the standard genetic techniques. The gene of interest can include any nucleotide sequence with an open reading frame (no stop codons) up to the 3′ end of the gene and, for the purposes of this invention, the end of the anchor (spacing) sequence.

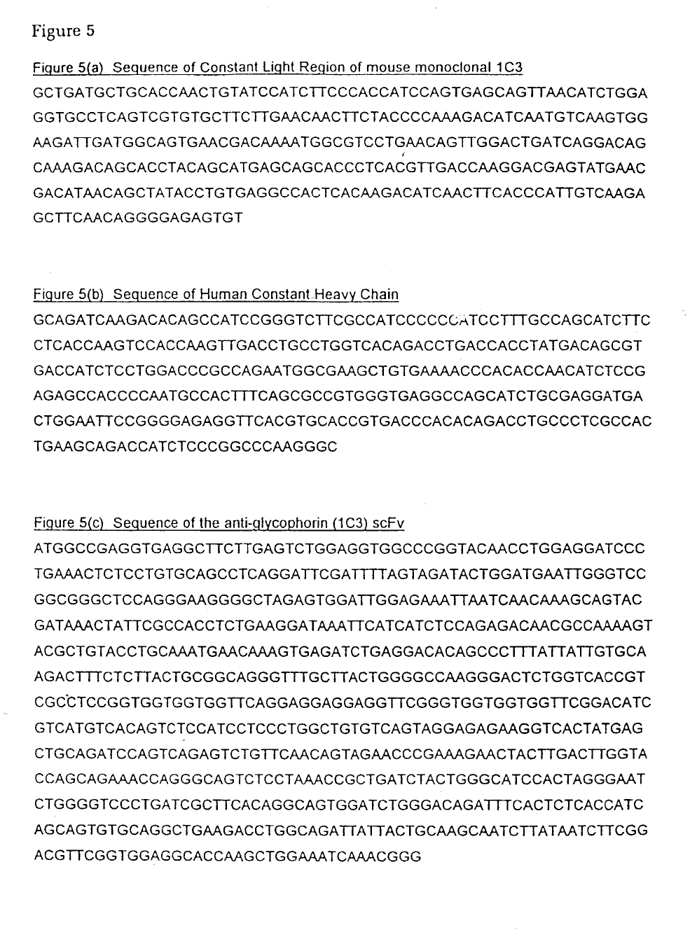

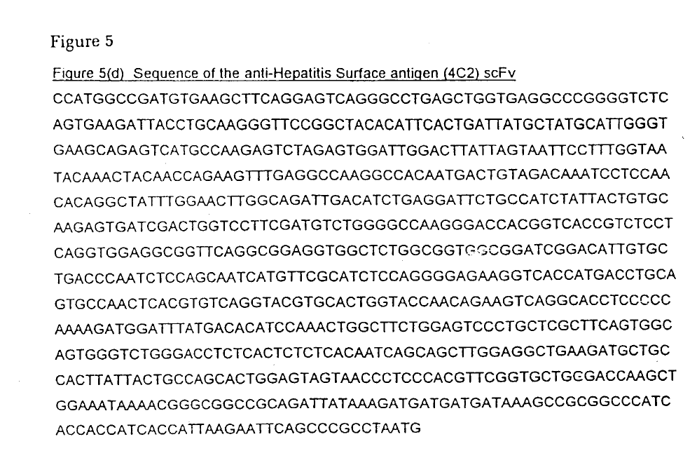

In a preferred embodiment the gene(s) of interest is a nucleotide sequence coding for i) a library of proteins or ii) a single protein. The protein can bind or react with a target molecule such as a protein, DNA, cell surface molecule, receptor, antibody, enzyme, hormone, virus, small molecule or any other molecules or complexes or derivatives thereof A nucleotide sequence coding for an anchor domain can be fused 3′ and in frame with the gene of interest. The anchor domain can be any of a series of polypeptide sequences sufficiently long to space the protein translated from the gene of interest a sufficient distance from the ribosome to allow correct folding of the molecule and accessibility to its cognate binding partner. In a preferred embodiment the anchor is the sequence coding for the octapeptide “FLAG” epitope: DYKDDDDK (SEQ ID NO:27), or any of the human or murine antibody constant domains. Preferably, the anchor is the constant domain from a mouse monoclonal antibody, such as constant domain 1C3 (see FIG. 5a). A further preferred anchor is the constant region from a human IgM antibody (see FIG. 5b).

The anchor sequence can be followed by a translation termination (stop) nucleotide sequence e.g. TAA or TAG. However, in some constructions it could be envisaged that no stop codons should be present to prevent recognition by release factors and subsequent protein release. In these, the anti-sense ssrA oligonucleotide sequence can be added to prevent addition of a C terminal protease site in the 3′ untranslated region that follows. The addition of sparsomycin, other similar compounds or a reduction in temperature also prevents release of the ribosome from the RNA and de novo synthesized protein.

The Expression System

Transcription/replication/mutation for the expression unit can be achieved by use of a rabbit reticulocyte lysate system (He and Taussig, 1997) or an E. coli S-30 transcription translation mix (Mattheakis et al., 1994; Zubay, 1973). For example, a DNA expression unit (detailed above) with a T7 promoter is treated with T7 RNA polymerase according to the manufacturer's instructions. The resulting RNA library reflects the diversity of the encoded genes. RNA dependent-RNA polymerases added for replication and mutation can be supplied either as purified enzyme or alternatively encoded as a distinct expression unit in a plasmid under control of a promoter such as T7 or SP6. The preferred enzyme is Qβ replicase although any enzyme with similar characteristics can be used. This step provides the increase in complexity of the library through mutation by the Qβ replicase. For RNA synthesis in eukaryotic cells the RNA is preferably capped, which is achieved by adding an excess of diguanosine triphosphate; however, the rabbit reticulocyte system from the commercial suppliers Promega and Novagen have components in the system to make the addition of capping compounds unnecessary. The transcription/translation mix or coupled system can be extracted from any cell; those most commonly used are wheat germ, mammalian cells such as HeLa cells, E. coli and rabbit reticulocytes. The coupled transcription translation system can be extracted from the E. coli mutator cells MUTD5-FIT (Irving et al., 1996), which bear a mutated DNAQ gene and therefore allow further random mutations introduced into DNA during replication as a result of proofreading errors. One preferred transcription/translation mix is the rabbit reticulocyte lysate. Addition of GSSG to the coupled system enhances correct folding of displayed proteins and therefore enhances subsequent selection to counter-receptors or antigens.

Mutation by Qβ Replicase

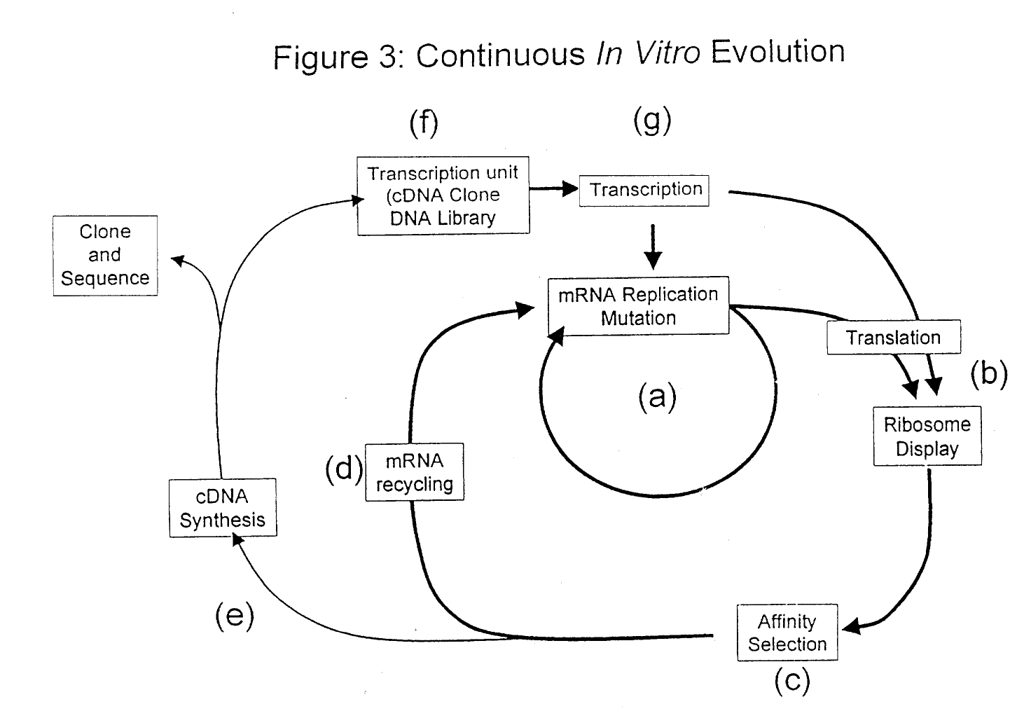

The Qβ replicase is included in the system for the replication and production of high levels of RNA incorporating random mutations (see FIG. 3). Multiple copies of a single-stranded RNA template are generated as a result of the action of Qβ replicase. These copies incorporate mutations and can themselves act as templates for further amplification by Qβ replicase as both RNA strands are equally efficient as templates under isothermal conditions.

Teaching in the art indicates that the complex and stable secondary and tertiary structures present in full length RNA from phages such as Qβ limit the access of ribosomes to the protein initiation sites. However, we have found that smaller RNA sequences are suitable for binding of replicases and therefore can be used instead of full-length templates. Preferred sequences are small synthetic RNA sequences known as pseudoknots (Brown and Gold 1995; 1996), which are compatible with amplification by Qβ replicase. In the context of the present invention, the use of pseudoknots can overcome the problems of ribosome access to the protein initiation sites whilst maintaining the binding sites necessary and sufficient for the Qβ replicase amplification of the RNA and sequences fused thereto.

In Vitro Translation and Ribosome Display

Several in vitro translation methods are known which can be either eukaryotic, such as rabbit reticulocyte lysate and wheat germ, or prokaryotic, such as E. coli. These are available commercially or can be generated by well known published methods. Translation of the mutated RNAs produces a library of protein molecules, preferably attached to the ribosome in a ternary ribosome complex which includes the encoding specific RNA for the de novo synthesized protein (Mattheakis et al., 1994). Several methods are known to prevent dissociation of the RNA from the protein and ribosome. For example, sparsomycin or similar compounds can be added; sparsomycin inhibits peptidyl transferase in all organisms studied, and may act by formation of an inert complex with the ribosome (Ghee et al., 1996). Maintaining high concentrations of magnesium salts and lowering GTP levels may also contribute to maintaining the ribosome/RNA/protein complex, in conjunction with the structure of the expression unit detailed above. A preferred means to maintain the ternary ribosome complex is the omission of the translation stop codon at end of the coding sequence.

In addition, there are preferred requirements for the correct folding of the molecules in the two systems. For prokaryotes, protein disulphide isomerase (PDI) and chaperones can be used as well as a C terminal anchor domain to ensure the correct folding. The latter is required, as prokaryotic proteins are released from the ribosomes prior to folding (Ryabova et al., 1997) and therefore, in situations in which the peptide is anchored to the ribosome the entire protein needs to be spaced from the ribosome. In contrast, in eukaryotic systems, the protein is folded as it is synthesized and has no requirement for the prokaryote PDI and chaperones to be added; however, we have found that addition of a specific range of GSSG concentrations is beneficial to the library selection by the enhanced display of correctly folded proteins on the ternary ribosome complexes.

Selection and Competitive Binding

Successive rounds of RNA replication produce libraries of RNA molecules which, on translation, produce libraries of proteins. A target molecule-bound matrix (for example antigen-coated Dynabeads®) can be added to the reaction to capture ternary ribosome complexes. The individual members in the library compete for the antigen immobilized on the matrix (Dynabeads®). Molecules with a higher affinity will displace lower affinity molecules. At the completion of the process the complexes [RNA/ribosomes/protein] attached to matrix (Dynabeads®) can be recovered, cDNA can be synthesized from the RNA in the complex and cloned into a vector suitable for high-level expression from the encoded gene sequence.

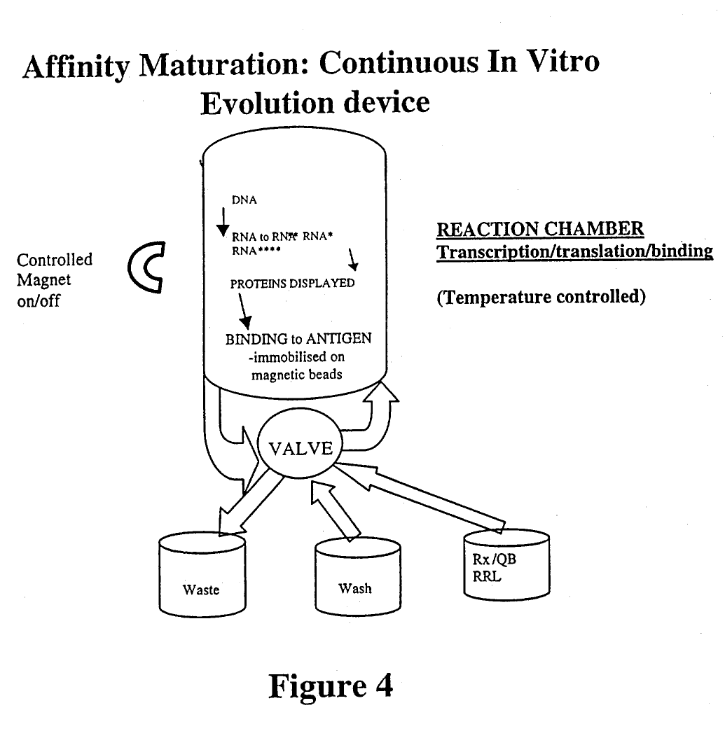

A recycling flow system (Spirin et al., 1988) can be applied to this Continuous in vitro Evolution (CIVE) system using a thermostated chamber to ensure supply of substrates (including ribosomes) and reagents and removal of non-essential products. All processes of CIVE can take place within this chamber, including: coupled transcription and translation, mutating replication, display of the de novo synthesized protein on the surface of the ternary ribosome complex, and competitive binding of the displayed proteins on the ternary ribosome complex to antigen to select those with the highest affinity binding (FIG. 4). The unbound reagents, products and displayed proteins are removed by flushing with washing buffer, and the bound ternary ribosome complexes are dissociated by increasing the temperature and omitting the magnesium from the buffer. This is followed with the addition of all the reagents necessary to carry out all the above steps except the washing buffer steps. Methods are available to prevent dissociation of the RNA from the protein and ribosome, such as the addition of sparsomycin or similar compounds. Maintaining specific concentrations of magnesium salts and lowering GTP levels may also contribute to maintaining the ribosome/RNA/protein complex, as well as reducing the reaction temperature or omitting translational stop codons. By using vessels whose temperatures are controlled, combined with a continuous flow capability, RNAs from selected ribosomes can be dissociated from the ribosomes and further replicated, mutated and translated, as the concentration of reagents important for the maintenance of the ribosome/RNA/protein complex such as sparsomycin, Mg etc are varied. FIG. 4 depicts the design of such a device.

The present invention will now be more fully described with reference to the following non-limiting Examples.

### EXAMPLES

### Example 1

### Expression and Purification of Recombinant Qβ Replicase

Cloning and Expression

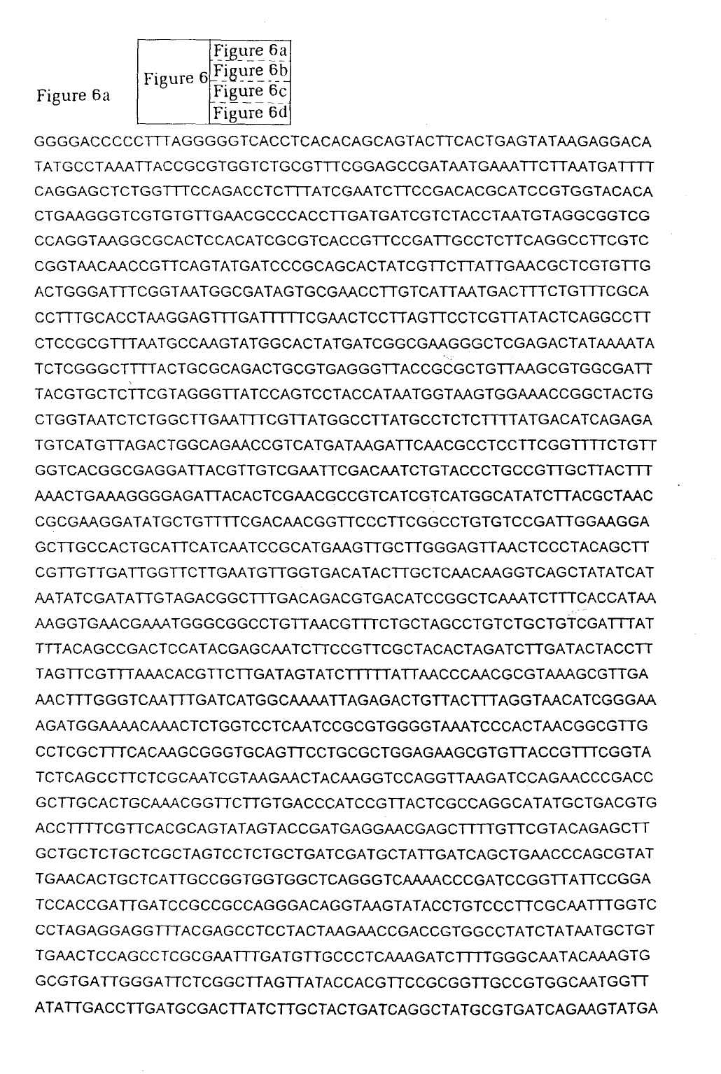

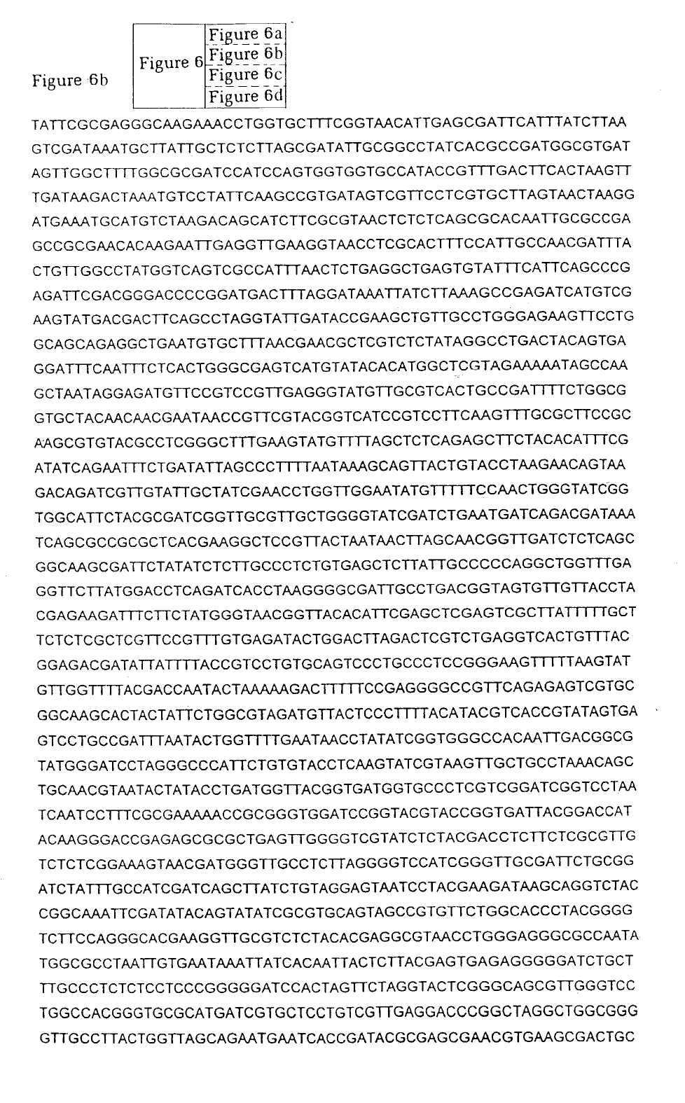

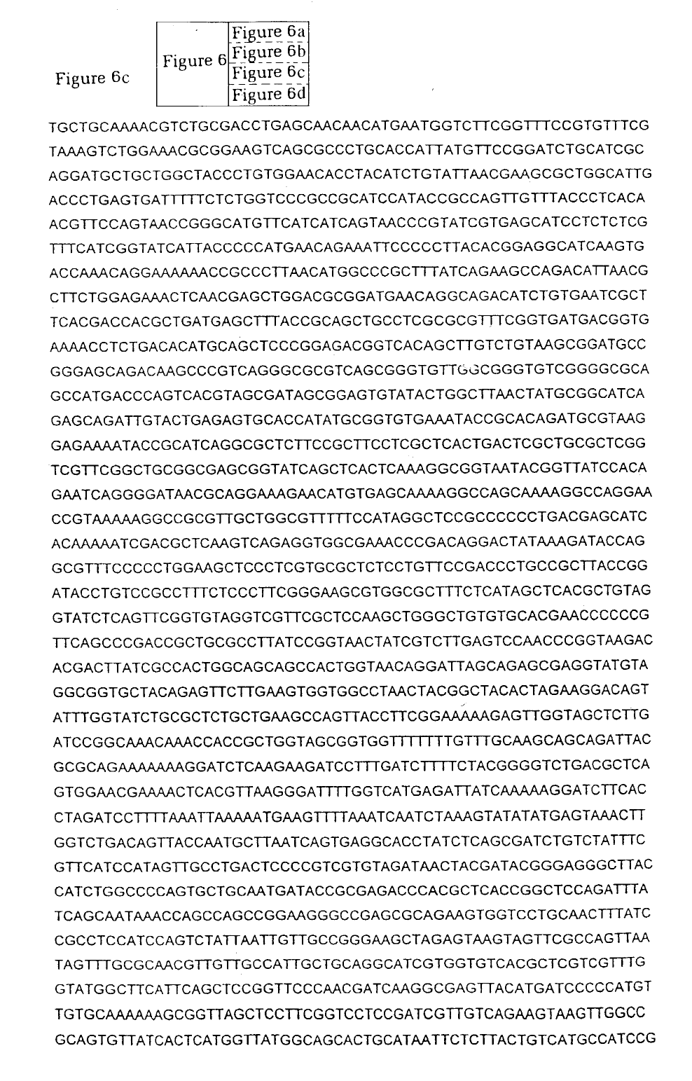

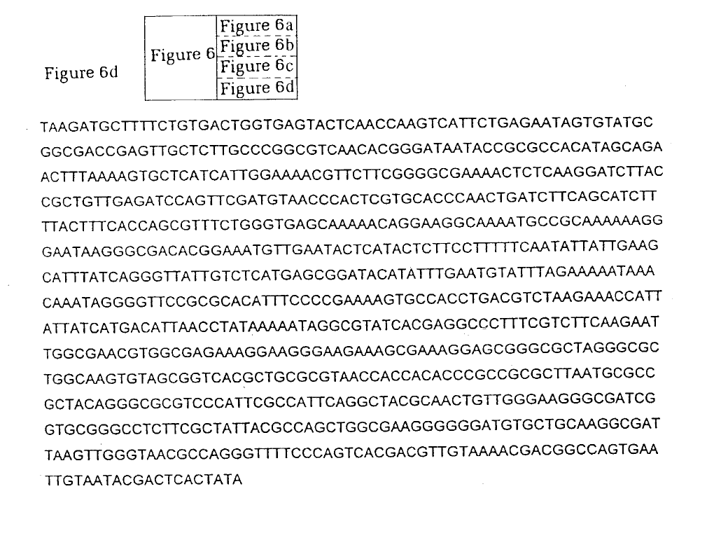

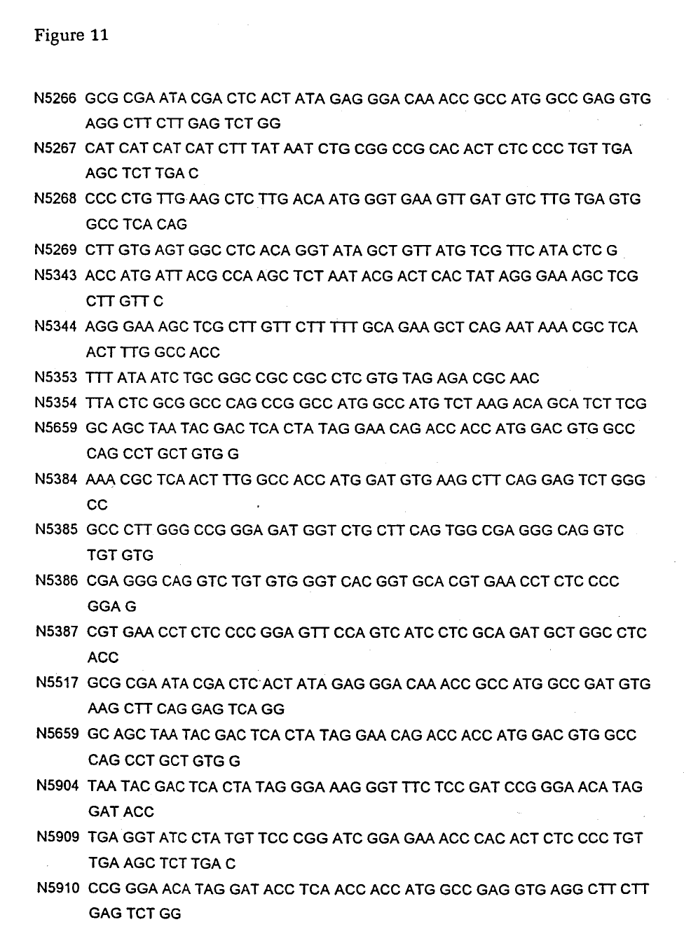

The Qβ replicase coding sequence was amplified by PCR from the plasmid pBRT7Qβ, a pBR322 based construction (briefly described in Barrera et al., 1993) that was designed to allow the preparation of infectious RNA by transcription using T7 RNA polymerase in vitro, being a cDNA copy of the RNA genome of phage Qβ. The sequence of pBRT7Qβ is shown in FIG. 6. Nucleotide no. 1 is the first nucleotide of the Qβ replicase sense strand. The oligonucleotides used as primers to amplify the Qβ replicase encoded sites for restriction enzyme digestion by the enzymes EcoRI and Not I and the sequences are shown in FIG. 11.

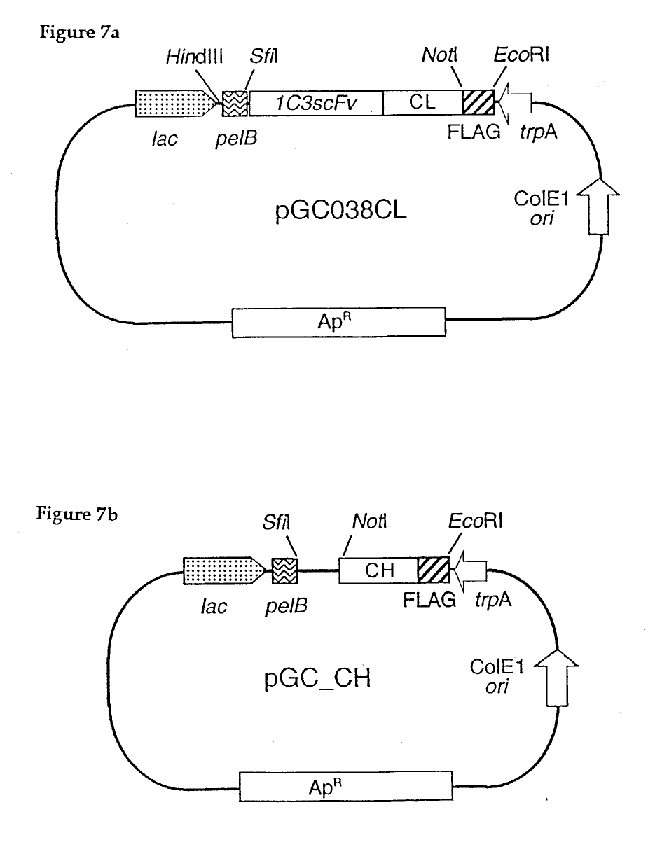

The PCR products were purified using any one of the commercial products available for this purpose (for example, Bresatec). The purified DNA was cloned into the EcoRI and NotI sites of the vector pGC (FIG. 7a) using standard molecular biology techniques. The vector pGC and expression of recombinant protein therefrom has been described in the literature and is incorporated herein by reference (Coia et al., 1996). The processes of the PCR amplification and cloning of the Qβ replicase gene into vectors, and transformation into E. coli for expression of the enzyme will be known to those skilled in the art, as will be the expression of the Qβ replicase gene in pGC, which was induced by adding 1 mM ispropylthiogalatoside (IPTG) to the culture medium.

Expression and purification of the Qβ replicase gene in the pBR322 based vector with the promoter PL was performed as detailed below. The rep14 Billeter strain was supplied by Christof Biebricher, Max Planck, Gottingen. The E. coli strain was grown in a 20 l fermentor in 2% nutrient broth, 1.5% yeast extract, 0.5% NaCl, 0.4% glycerol, 100 mg/l ampicillin with good aeration at 30° C. to an optical density of 2 (660 nM). After raising the temperature to 37° C., aeration was continued for 5 h. The cells were chilled on ice and harvested by centrifugation (yielding about 180 g wet cell mass).

Purification of Qβ Replicase

Buffer A: 0.05M Tris HCl-buffer (pH 7.8), 1 mM mercaptoethanol, 20% v/v glycerol, 100 mg/l ampicillin.

Buffer B: 0.05M HEPES Na-buffer (pH 7.0), 1 mM mercaptoethanol, 20% v/v glycerol.

50 g harvested E. coli were homogenized with 100 ml 0.05M Tris HCl buffer (pH 8.7) 1 mM mercaptoethanol in a high-speed blender. Lysozyme and EDTA were added to final concentrations of 100 μg/ml and 0.5 mM, respectively, and the solution was gently stirred at 0° C. for 30 min. 12 ml 8% Na deoxycholate, 0.24 ml phenylsulfonylfluoride (20 mg/ml in propanol-2), 0.15 ml Bacitracine (10 mg/ml), 0.15 ml 0.1M benzamidine, 3.3 ml 10% Triton-X-100 were added and the solution adjusted with MgCl₂ to 10 mM final concentration. The high viscosity was reduced by blending at high speed. Solid NaCl was added to a final concentration of 0.5M and 4.8 ml 0.3% polyethyleneimine (pH 8) was added with stirring. After stirring for 20 min at 0° C. the suspension was centrifuged for 30 min. at 10,000 rpm (GSA rotor). After dilution of the supernatant with 5 volumes Tris HCl buffer (pH 8.7) 1 mM mercaptoethanol, 100 ml DEAE cellulose slurry (Whatman DE52, equilibrated with buffer A) was added and slowly stirred at 0° C. for 20 min. After a 40 min. incubation without stirring, the supernatant was decanted from the sediment and discarded. The sediment was suspended in buffer A, poured into a glass column of 1 cm diameter, washed with 400 ml Tris HCl buffer (pH 8.7) 1 mM mercaptoethanol, and eluted with 250 ml buffer A+180 mM NaCl; fractions were collected. The fractions were assayed for the presence of Qβ replicase using the following binding assay.

Enzyme Location Assay: Binding of Biotinylated RNA to Qβ Replicase

This is a non-radioactive assay developed to detect replication enzymes, which relies on biotin-labelled RNA bound to enzyme being retained on positively charged membranes, whereas, free biotin-labelled RNA under the same conditions is not retained on the membrane. DNA and RNA were labelled with psoralen-biotin (Ambion) according to the manufacturer's instructions. The labelled RNA was then added to the column eluate (sample fractions) as indicated in the assay below to detect the location of Qβ replicase.

The following was mixed in an Eppendorf tube:

10 μl column eluate fractions

10 μl 0.5M Tris HCl (pH 7.4) containing 120 mM MgCl₂

10 μl 2 mM ATP

10 μl 5 mM ATP

10 μl˜100 ng/ml psoralen-biotin labelled probe RNA

50 μl water

The reaction mix was incubated at 37° C. for 1 min.

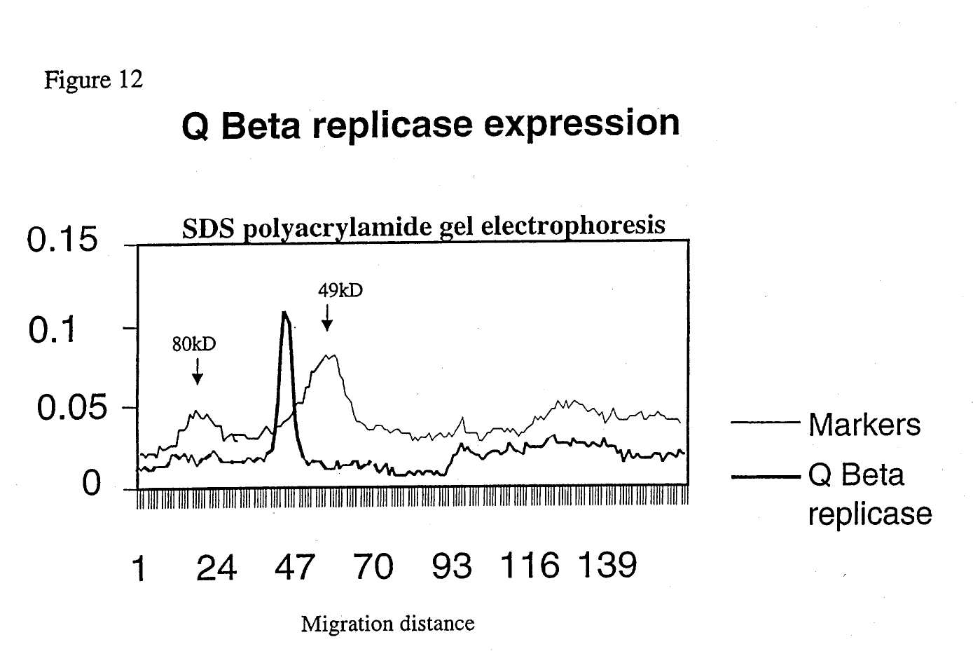

The reaction mixtures were dot blotted onto nylon membrane, e.g. hybond N, (only RNA or DNA binds to the enzyme—Qβ replicase will be retained on the membrane), washed with the 50 mM Tris HCl pH 7.4 containing 12 mM MgCl₂, UV cross linked onto the nylon membrane in the Stratalinker on the automatic setting. The BrightStarm BioDetect™ kit was used for the detection of the biotinylated nucleic acid attached to the nylon membrane. FIG. 12 shows the assay of the eluted fractions from the DE52 column.

The active fractions were pooled, diluted with one volume buffer A and applied to a 35 ml column of DEAE-Sepharose FF, equilibrated to buffer A+0.1M NaCl. The enzyme was eluted with a linear gradient of 0.1-0.4M NaCl in buffer A. The active fractions were pooled, the enzyme precipitated by addition of solid (NH₄)₂SO₄ (39 g/100 ml solution), collected by centrifugation and dissolved in 4 ml buffer B.

The enzyme was diluted until the conductivity was less than that of buffer B+0.2M NaCl and applied to a 100 ml column of Fractogel EMD SO₃ equilibrated with buffer B, and eluted with a linear gradient (2 times 500 ml) of 0.2-0.8˜M NaCl in buffer B. The active peaks, eluting at about 0.65M NaCl, were pooled, precipitated with solid (NH₄)₂SO₄ (39 g/100 ml solution), collected by centrifugation, and dissolved in 10 ml buffer A+50% glycerol. The solution was stored at −80° C.

The following steps were performed at small scale according to Sumper & Luce (1975). 4 mg Qβ replicase were applied to a 1.6×14.5 cm column of QAE-Sephadex˜A-25 equilibrated with buffer A (diluted or dialyzed to remove salt), and eluted with a 2×200 ml gradient of 0.05-0.25M NaCl in buffer A. The two clearly separated peaks of core and holoenzyme were pooled, diluted 1:1 with buffer A and applied to QAE-Sephadex columns, 2 ml for core, 6 ml for holo replicase, respectively, washed with buffer A+50% glycerol, and the replicase was eluted in concentrated form with buffer A+50% glycerol+0.2 M (NH₄)₂SO₄. The active fractions were stored at −80° C. Care was taken to avoid contamination of the equipment with RNA.

### Example 2

### Cloning of Qβ Replicase into the Eukaryotic Expression Vector pcDNA3.1

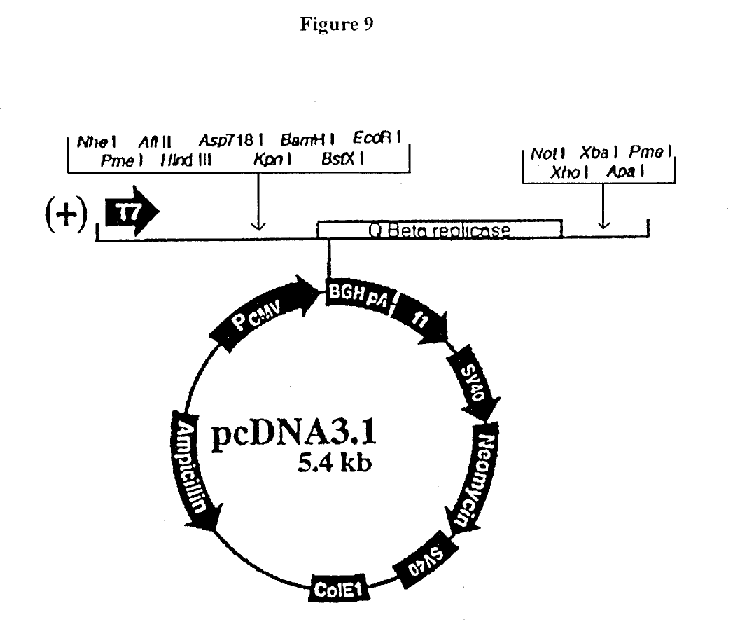

Qβ replicase coding sequence was cloned into the eukaryotic expression vector pcDNA 3.1 (FIG. 9) to produce the vector named pcDNAQβ. This vector was used for the expression of Qβ replicase in situ in the coupled transcription/translation system and concomitant replication/mutation of target RNA. Sequence of oligonucleotides used as primers in PCR amplification of Qβ replicase for cloning into the EcoRI and NotI restriction sites in the eukaryotic expression vector pcDNA3.1 were:

(SEQ ID NO:28)

| | |
|---|---|
| # 5352 | 5′TCTGCAGAATTCGCCGCCACCATGTCTAAGACAGCATCTTCG |

(SEQ ID NO:29)

| | |
|---|---|
| # 5350 | 5′TTTATAATCTGCGGCCGCTTACGCCTCGTGTAGAGACGC |

The coding sequence for the Qβ replicase b subunit was cloned into the pcDNA3.1 by standard molecular biology techniques (Sambrook et al., 1989). The cloned sequence was confirmed by DNA sequence analysis. Expression of the Qβ replicase in the rabbit reticulocyte coupled transcription/translation system was followed by the detection of biotinylated lysine (Transcend™, Promega) incorporated into the de novo synthesized Qβ replicase in the standard transcription/translation reaction as suggested by the commercial suppliers of the coupled trancription translation kits (Promega and Novagen) and the supplier of Transcend™ (Promega). At the completion of the incubation step of the coupled reaction, 20 μl of the reaction was heated to 90° C. with 2 ml of 10×SDS sample buffer and the samples subjected to SDS polyacrylamide gel electrophoresis (SDS-PAGE). This was followed by Western blotting and the de novo synthesized biotinylated Qβ replicase bands detected with Transcend™ kit detection reagents. The results of this expression are shown in the gel scans of FIG. 12 where it can be seen that Qβ replicase has been synthesized shown by the biotinylated band at the correct size on the gel.

We then undertook coupled transcription/translation reactions with the 1C3 template (Example 3) but also expressing the Qβ replicase from pcDNA3.1 in the same reaction. The Qβ replicase synthesized in situ from the expression vector pcDNAQβ resulted in the increased synthesis of the 1C3 scFv in the coupled system in the presence of 0.5 mM manganese chloride; measured by incorporation of biotinylated lysine (FIG. 12b) as described above. The presence of the manganese chloride has previously been shown to relax the dependence of the Qβ replication activity on transcription/translation factors.

### Example 3

### Construction by PCR of DNA Templates for Transcription

DNA sequences were amplified by standard and well-described techniques (Polymerase Chain Reaction [PCR] with specifically designed oligonucleotide primers, splice overlap extension, restriction enzyme digests, etc.) using either Taq, Tth, Tfl, Pwo or Pfu polymerase, according to the supplier's instructions, and using either an FTS-1 thermal sequencer (Corbett Research), a PE2400 (PerkinElmer) or a Robocylcer® gradient 96 (Stratagene). A list of oligonucleotide primers used is given in FIG. 11. Products were gel purified using BresaClean™ (Bresatec) or used directly in coupled transcription and translation reactions.

DNA sequences were amplified from starting templates that had been cloned into either vector pGC038CL (FIG. 7a) or vector pGC_CH (FIG. 7b), which provided an extension to the 3′ terminus of the construct. This extension was either a constant region from a mouse monoclonal antibody (1C3; Sequence FIG. 5a) or a constant region from a human IgM antibody (Sequence FIG. 5b). Forward (sense) primers (N5266 for the anti-GlyA scFv; N5517 or N5384, N5344 and N5343 for the anti-HepB scFv) used for amplification provided a transcriptional initiation site as well as a translational initiation site and ribosome binding site. Reverse (antisense) primers (N5267 for the mouse constant region; N5385 for the human constant region) did not contain stop codons, which allows the mRNA-ribosome-protein complex to remain associated. Both forward and reverse primers provided restriction enzyme sites (specifically SfiI and NotI, respectively) which enabled cloning of generated fragments.

Any of several promoter sequences for DNA dependent RNA polymerase can be used to direct transcription; however, the following sequences were the two preferred (these include translational initiation sequences; see below):

(SEQ ID NO:30)

| | |
|---|---|
| a) | GCGCGAATACGACTCACTATAGAGGGACAAACCGCCATGGCC |

(SEQ ID NO:31)

| | |
|---|---|
| b) | GCAGCTAATACGACTCACTATAGGAACAGACCACCATGGCC |

These sequences have directed transcription by T7 DNA dependent RNA polymerase to produce RNA transcripts in two alternative formats of coupled transcription/translation systems.

Sequences encoding ribosome binding sites are known and have been included in the template upstream of any one of the sequences of the molecules of interest for ribosome display encoding either the scFv binding to glycophorin (1C3; FIG. 5c) or the scFv binding hepatitis B surface antigen (4C2; FIG. 5d). The same sequences have been included in the template upstream of any other sequences of interest for ribosome display (eg CTLA-4-based library sequences).

### Example 4

### Coupled Transcription/Translation and Ribosome Display in Rabbit Reticulocyte Lysate Cell Free System

Transcription and translation was carried out in siliconized RNase-free 0.5 ml tubes (Ambion) using the TNT T7 coupled transcription/translation system (Promega) containing 0.5 mM magnesium acetate, 0.02 mM methionine and 3 mM oxidized glutathione (GSSG) (see Example 6, below), and the mixture was incubated at 60° C. for 90 min. In some reactions, up to 10 mM reduced glutathione was also added. In reactions containing Qβ polymerase, the mixture also contained manganese chloride to a final concentration of 0.5 mM. After transcription and translation, the mixture was diluted with PBS and treated with DNaseI to remove any remaining starting DNA template. This was achieved by the addition of 40 mM Tris (pH 7.5), 6 mM MgCl₂, 10 mM NaCl, and DNase I (Promega), followed by incubation at 30° C. for a further 20 min.

### Example 5

### Selection of Ribosome Ternary Complex Displayed Proteins Against Antigens Using Dynabeads®

Tosylactivated magnetic beads (Dynal) were coupled to GlycophorinA (GlyA; Sigma), HepatitisB Surface Antigen (HepB SA; BiosPacific, Emeryville, Calif. USA) or bovine serum albumin (BSA; Sigma) according to manufacturer's instructions. Where Streptavidin magnetic beads were used, these were coupled (according to manufacturer's instructions) to antigens (as shown above) which had been biotinylated using EZ-Link Sulfo-NHS-LC-Biotin (Pierce) according to manufacturer's instructions.

In order to select specifically binding mRNA-ribosome-protein complexes, 2-3 μl of antigen coupled (tosylactivated or streptavidin coated) magnetic beads were added to the final translation mixture and placed on a plate shaker (Raytek Instruments) at room temperature for 90 min with gentle shaking to prevent settling of the beads. The beads were recovered using a magnetic particle concentrator (Dynal) and these were washed three times with cold phosphate buffered saline (PBS) pH 7.4 containing 1% Tween and 5 mM magnesium acetate. The beads were then washed once with cold sterile water, and finally, resuspended in 10 μl of sterile water.

For the synthesis of cDNA from selected complexes, 2 μl of the final bead suspension was used in an RT-PCR reaction using either the Access RT-PCR system (Promega) or the Titan One Tube RT-PCR system (Boehringer Mannheim) according to manufacturer's instructions. The primers used for this reaction included the original forward (sense) primer (used to generate the starting template DNA primers; N5266 for the anti-GlyA scFv; N5517 or N5384, N5344 and N5343 for the anti-HepB scFv) and a negative (antisense) primer which was upstream of the original primer (N5268 and N5269 for mouse constant region constructs; N5386 and N5387 for human constant region constructs). In some cases, shorter primers (N5941 and N5942 for the anti-GlyA scFv-constant light region construct) were used to recover panned RNA templates.

For further cycles of selection, this DNA was gel purified (in some cases, simply diluted) and incorporated into a further PCR using the forward and reverse primers which had been present in the original PCR to generate the starting DNA template. This new template could then be used in further rounds of transcription, translation and selection as described above since it contained the appropriate initiation sites and is of the same length as the template in the first selection.

In order to show that the method described above can be used to select specific molecules, a single chain Fv (scFv) fused to a mouse constant light chain region which specifically binds to GlyA was amplified using primers which would allow the addition of a T7 transcriptional initiation site and a ribosome binding site. This template (T7-scFv) was used in a coupled transcription/translation reaction as described above and then split into three and mixed with either HepB SA, GlyA or BSA coupled magnetic beads. The beads were washed (as described above) and recovered mRNA-ribosome-protein complexes were used to synthesize cDNA. The results of this experiment showed the presence of a product of the correct size in each lane. The non-specific binding observed in the HepB SA and BSA lanes is probably due to aggregation of products synthesized during translation. It has been observed by others that only a proportion of products synthesized using the reticulocyte lysate are in a properly folded and active form. This problem was addressed in Example 6 below.

The GlyA specific product from this experiment was gel purified and re-amplified by PCR in order to synthesize more template for a further round of transcription, translation and selection. A second round of panning showed predominantly a specific product in the sample probed with GlyA coupled magnetic beads. This showed that by the second round of selection, the products recovered were specific for GlyA.

### Example 6

### Effect of Adding Oxidized and/or Reduced Glutathione

In an attempt to induce a higher proportion of correctly folded products during in vitro transcription and translation, various concentrations of either reduced or oxidized glutathione were added to the reaction mixture. The template used for these reactions was the anti-GlyA T7-scFv (as described above) and selections were performed using GlyA coupled magnetic beads. This experiment showed that the amount of recovered product increased with increasing concentrations of oxidized glutathione up to 5 mM. A further increase to 10 mM had a detrimental effect on the yield of recovered product. A concentration of around 2 mM oxidized glutathione was included in most transcriptions and translations.

Later results revealed that a further addition of 5 mM and 10 mM reduced glutathione to the reaction already containing 2 mM oxidized glutathione showed that the addition of 5 mM glutathione appeared to allow better folding of the displayed anti-GlyA scFv leading to an increased amount of recovered product from the GlyA panning over the control pannings. Further decreasing the concentration of reduced glutathione to to 0.5 mM showed similar effects.

### Example 7

### Display of Mutant v-Domain (CTLA-4) Library on Ribosomes

In order to show that ribosome display could be used to select binding elements from a polypeptide library, a library of CTLA4 mutants was ligated into plasmid pGC_CH (FIG. 7b), which allowed the addition of a constant heavy domain. This library was then amplified by PCR using primers N5659 and N5385 (FIG. 11). Primer N5659 was used to add the necessary upstream transcriptional and translational initiation sequences. This PCR DNA was then used as template for transcription and translation in a coupled cell free translation system using the methods described in Example 4. To demonstrate binding of mutant CTLA ribosome complexes, panning was performed using Hepatitis B surface antigen (HBSA), GlycophorinA (GlyA) and Bovine Serum Albumin (BSA) coated Dynabeads®. RNA attached to bound complexes was then recovered by RT-PCR. The methods used for panning, selection and recovery was as described previously (Example 5).

Products corresponding approximately to the size of CTLA4 based mutants were recovered and showed that the CTLA4 library contained DNA encoding proteins which specifically bind HBSA, GlyA and BSA. These products were cloned into the vector pGC_CH (FIG. 7b) for DNA sequencing and expression of soluble products. Sequencing using standard methods (BigDye Terminator Cycle Sequencing; PE Applied Biosystems CA) showed that CTLA4-based specific inserts were present. Furthermore, expression analyses using ELISA showed that specifically reactive proteins were being expressed by the recombinant cultures. In these assays, recombinants which had been isolated by panning using GlyA-coated Dynabeads® and screened by ELISA using GlyA-coated plates, gave stronger signals than similarly tested recombinants which had been isolated by panning using BSA-coated Dynabeads®.

### Example 8

### Effect of Including Qβ Replicase in Coupled Transcription and Translation

In a attempt to increase both the yield of products and the rate of mutagenesis in products during in vitro translation, Qβ replicase (in either of two forms) was added to the reaction mixture. The replicase was included as either a purified Qβ replicase protein or as a gene template under the control of a T7 transcriptional promoter (pcDNAQβ) which could be simultaneously synthesized during the coupled transcription/translation reaction. The template used for this reaction was again the anti-GlyA T7-scFv (as described above) and selections were performed using GlyA coupled magnetic beads. These experiments showed that the amount of recovered GlyA reactive product increased (over the no Qβ replicase control) with the addition of purified Qβ replicase and, to a lesser extent, with the addition of Qβ replicase-encoding genomic template (pcDNAQβ).

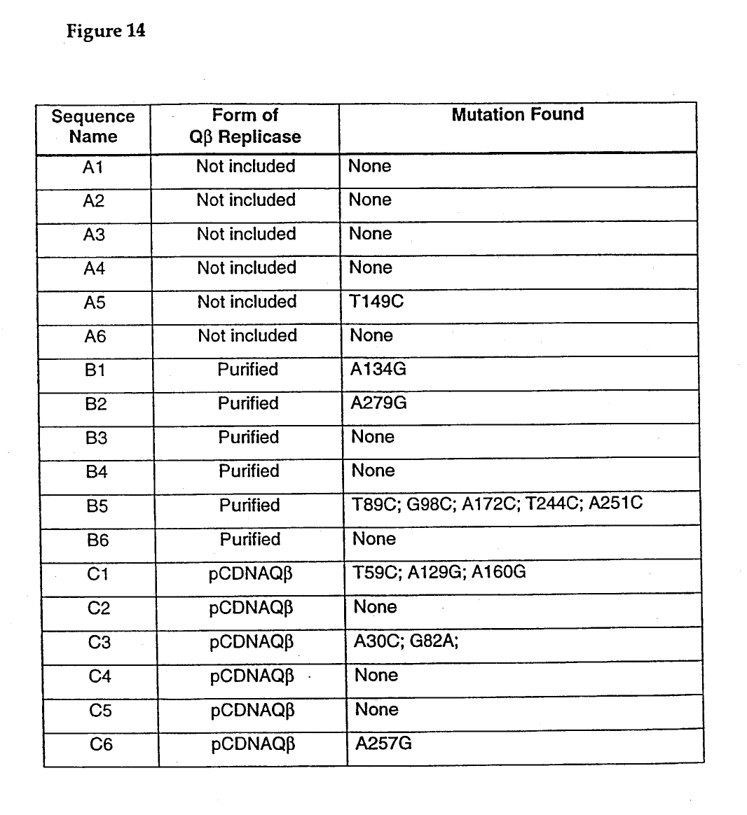

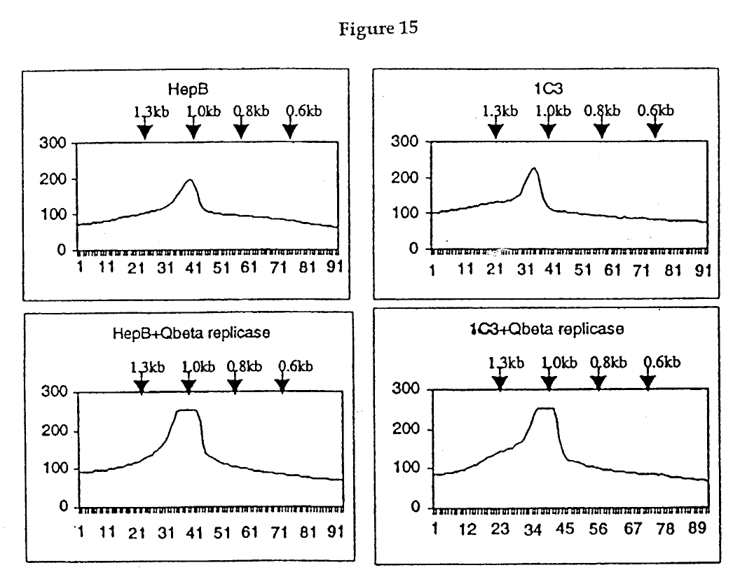

In order to determine whether mutations had been inserted into the scFv sequence, the main product from each lane was gel isolated and purified. The DNA was digested with SfiI and NotI and ligated into similarly digested pGC vector and transformed into E. coli using standard protocols. DNA was isolated from recombinants from each series and six random clones from each series were subjected to DNA sequencing using standard methods (BigDye Terminator Cycle Sequencing; PE Applied Biosystems CA). Approximately 280 bases were sequenced from each clone and FIG. 14 shows the number and the position of mutations in these sequences. This experiment showed the introduction of an increased number of mutations after transcription and replication in the presence of Qβ replicase (in either of the forms used).

### Example 9

### Addition of Artificial Qβ Sequences

In an attempt to increase the efficiency of Qβ replicase activity, specific Qβ binding sites were added to both the 5′ and 3′ ends of the anti-GlyA T7-scFv template by PCR. This new template (amplified with primers N5904 and N5910 [sense] and N5909 [anti-sense]; FIG. 11) was used in a coupled transcription/translation reaction that included Qβ replicase as either a purified Qβ replicase protein or as a gene template under the control of a T7 transcriptional promoter which could be simultaneously synthesized during the coupled transcription/translation reaction. Selections were performed using HepB, GlyA or BSA coupled magnetic beads and products were recovered after RT-PCR. The presence of artificial Qβ stemloop sequences (i) did not have an adverse effect on coupled transcription, translation and selection and (ii) in most cases increased the amount of products recovered by RT-PCR after selection.

### Example 10

### Replication of Anti Glycophorin scFv Transcripts by Qβ Replicase in the Coupled Transcription/Translation Rabbit Reticulocyte System

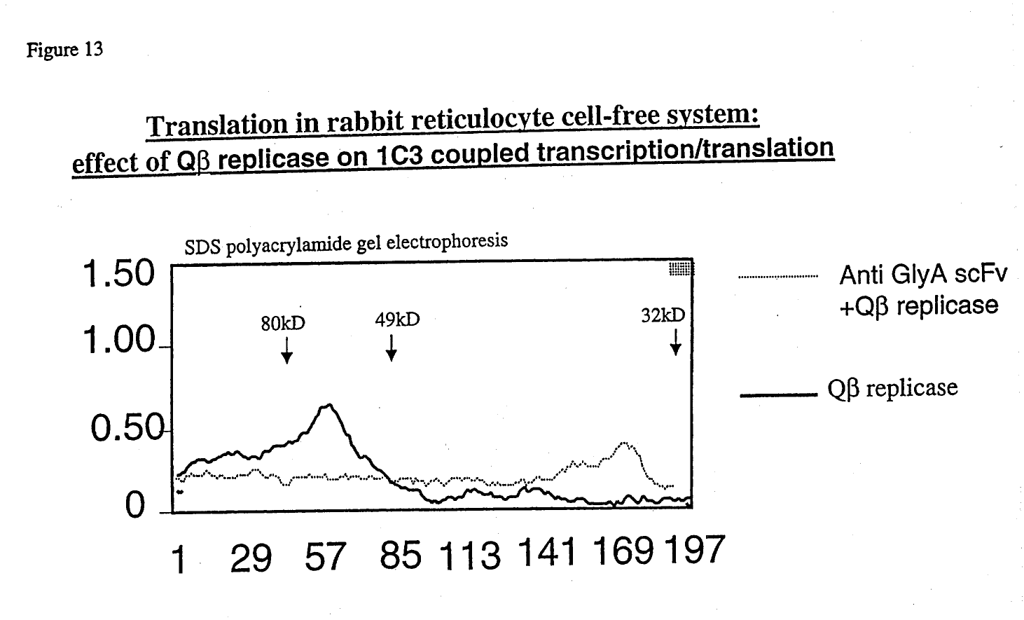

The T7-1C3 and T7-4C2 scFv templates for ribosome display were constructed as described in Example 3 and subjected to coupled transcription/translation, under the following conditions. Standard coupled transcription/translation reactions were modified by the addition of Qβ replicase (purified as detailed in Example 1). In a standard 20 μl reaction, 1 ml of 20 μg/ml enzyme was added. Previously, we have compared the effect of Qβ replicase concentration on replication of anti GlyA 1C3 scFv and anti Hepb 4C2 scFv in the coupled system, and observed that 1 ml of this sample provided the optimum replication. Manganese chloride was added to a final concentration of 0.5 mM, as this has been shown in published reports to decrease the requirement for transcription/translation factors. Reactions were allowed to continue for 2 hrs at 37° C. The replicated transcripts were analyzed by RT-PCR after removing DNA template by DNAase I digestion in 40 mM Tris-HCl pH 7.5, 6 mM MgCl₂, 10 mM NaCl at 30° C. for 20 min. Standard phenol extraction was used to remove DNAaseI and other proteins. Samples were ethanol precipitated and the RNA precipitate was dissolved in RNAase-free water. The RNA was assayed by RT-PCR using primers specific for each template (see Example 3), and the PCR products (DNA) were compared by agarose gel electrophoresis. The DNA bands were visualized by staining with ethidium bromide. The agarose gel was subjected to densitometry by scanning the digitized image with the gel-pro analyzer commercial software. FIG. 13 shows the densitometer traces of the agarose gel from which it can be seen that in the sample containing the purified Qβ replicase there is an increase in the amount of template produced.

### Example 11

### Replication and Mutation of Anti Glycophorin scFv and Anti Hepatitis B scFv by Qβ Replicase from T7 Polymerase Transcripts: Qβ Replicase Mutates Transcripts During RNA Dependent RNA Replication

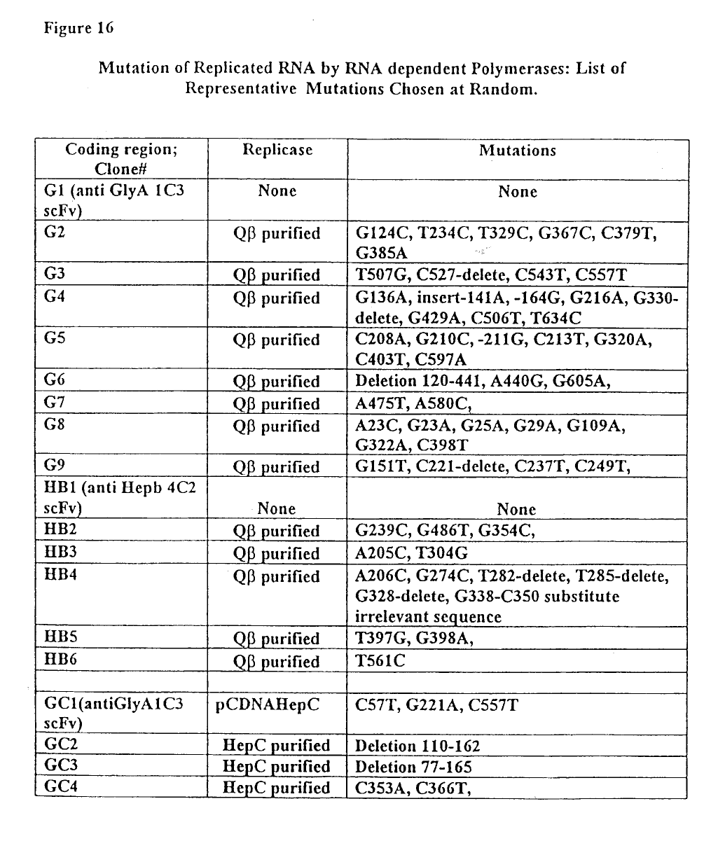

Coupled transcription/translation reactions, as detailed in previous examples, were supplemented with Qβ replicase purified enzyme to replicate and mutate the T7 DNA dependent RNA polymerase transcribed anti GlyA 1C3 scFv RNA. Following the transcription/replication/mutation/translation incubation, the sample was treated with DNAaseI, and this enzyme was removed as detailed in Example 10. The purified RNA was then used as the template for RT-PCR reactions with anti GlyA 1C3 scFv-specific primers in the reaction as detailed in Example 3. The thermostable polymerases used in these reactions were one of the high fidelity vent, pfu polymerase enzymes used in accordance with the manufacturer's instructions. The PCR reaction products were purified with one of the commercially available kits, as noted before, and the purified DNA was ligated into the commercially available plasmid pCRscript and transformed into competent E. coli XL1Blue cells, using standard molecular biology techniques. The transformation reactions were plated onto YT-agar plates containing X-gal. After overnight incubation white colonies (E. coli with plasmids containing DNA inserts in the multi-cloning site) were picked and grown overnight at 37° C. in 5 ml of YT broth containing 100 μg/ml ampicillin. DNA was extracted from each of the cultures with a commercial kit (Qiagen), according to the manufacturer's instructions. The purified DNA was analyzed by DNA sequencing; the sequencing results are displayed in FIG. 16. This table shows mutations in a random sample of sequences representing a minute sampling of mutations and sequence variation in the whole Qβ replicase replication/mutation reactions.

### Example 12

### Predicted Secondary Structure of Template RNA

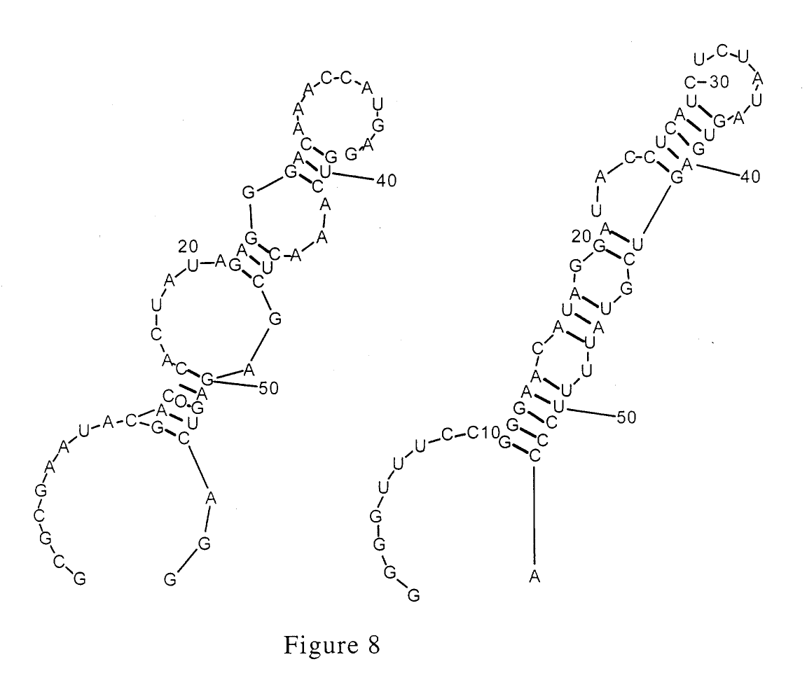

The RNA sequences and putative secondary structures preferred by Qβ replicase for its RNA templates have been reported (Zamora et al., 1995). To determine whether these or related preferential structures exist in the templates for the continuous in vitro evolution the upstream untranslated sequences, T7 promoter sequences, the sequences encoding the 1C3 gene, the constant light anchor region gene, the anti hepb 4C2 scFv gene and the IgM human constant heavy anchor region gene were analyzed with the Mfold program (Zucker et al, 1991) and compared to the Qβ replicase preferred structures (as shown in FIG. 8). From this comparison it can be seen that the 1C3 scFv has been identified to have internal RNA secondary structure mimicking the M site structure of Qβ replicase, as does the CL anchor region and shows similarity to the preferred synthetic sequence reported by Zamora et al., 1995. This may explain the preferred replication of the anti GlyA 1C3 scFv CL template to that of the anti Hepb 4C2 scFVCH3 by Qβ replicase (see Example 3). Therefore the CL region gene is proposed as an anchor region for displayed molecules for coupled transcription/translation display and any mutagenesis as the RNA encoding this region promotes and enhances Qβ replicase replication and associated mutation of this region and its genetic fusions.

### Example 13

### Expression Protocol for pLysN-NS5B (83 kDa, pI˜9.05)

pLysN-NS5B is a bacterial (cytoplasmic) expression vector with a T7 promotor. NS5B is the non-structural HepC RNA-dependent RNA-polymerase. NS5B is fused to a LysN moiety at its N terminus which are separated by a Gly-Ser-Gly-Ser-Gly linker, 10 His residues, and followed by a Asp-Asp-Asp-Asp-Lys linker: GSGSGHHHHHHHHHHDDDDK (SEQ ID NO:32).

This plasmid was transformed into E. coli strain HMS174(DE3)pLysS and grown on 1YT/Amp_{100 μg/ml}/Chloramphenicol_{34 μg/ml} agar plates at 37° C. A single colony was selected and cultured in an overnight broth 1YT/Amp_{100 μg/ml}/Chloramphenicol_{34 μg/ml}) at 37° C. For expression, the overnight starter culture was subcultured by dilution to an A600=0.1 in 1YT/Amp_{100 μg/ml}/Chloramphenicol₃₄_{μg/ml} at 37° C. in 2L shake flasks at 120 rpm. The culture was grown until the A600 reached 0.8-1.0 and then induced with 1 mM IPTG, supplemented with Amp_{100 μg/ml} and expression allowed to proceed at 37° C. for 4-5 hours.

The culture was harvested and centrifuged at 5000 g in a prechilled rotor at 4° C. The wet weight of the harvested culture was measured and the cell pellet frozen at −80° C. Approximately 3-4 grams was produced (wet weight) per litre of cell culture.

Lysis and Purification Protocol

Extraction of the HepC RdRp (NS5B) was achieved by lysing the cells followed by conventional protein chemistry techniques.

To the frozen cell pellet 5 ml of Buffer C (made fresh) at 4° C. was added per gram of cell pellet. The mixture was stirred at 4° C. using a magnetic bead until the culture was completely resuspended. The culture was then sonicated with 11 bursts each of 10 seconds with 1 minute pause between each burst, while continually stirring with a magnetic bead throughout the sonication process. The sonicated cells were centrifuged at 75000 g at 4° C. for 20 minutes and the supernatant (lysate) recovered.

A 30% saturation of (NH₄)₂SO₄ was added to the lysate and then the mixture was centrifuged at 10,000 g for 15 minutes. This acted to eliminate some precipitated bacterial proteins. The pellet was discarded and, to the supernatant, a 50% saturation of (NH₄)₂SO₄ was added, and the mixture was centrifuged at 10,000 g for 15 minutes. This acted to precipitate the NS5B from a large proportion of E. coli proteins. The supernatant was discarded and the pellet resuspended in half the original volume with Buffer C. This suspension was dialysed against Buffer C at 4° C. overnight.

An aliquot from each step was analyzed on SDS PAGE to confirm partial purification of ˜90 kDa HepC RdRp band.

The dialyzed extract was loaded onto a cation exchange column with Hyper D “S” resin pre-equilibrated with Buffer C. The column was then washed with Buffer C until a stable baseline was achieved. Elution was performed with a step gradient of Buffer C with 1M NaCl. It was found that NS5B eluted at 600 mM NaCl concentration.

The eluted fractions were analyzed on a 10% SDS PAGE to confirm purification. NS5B was purified by this process to over 90% homogeneity with minor smaller molecular weight contaminating proteins.

The purified NS5B was concentrated by 50% saturation with (NH₄)₂SO₄ and resuspension in a volume of Buffer C (with Tris pH 7.4) sufficient to redissolve the pellet. This was then dialyzed in the same buffer to eliminate the (NH₄)₂SO₄.

The purity of the NS5B was such that further purification by size exclusion chromatography on a preparative Superose 12 column in Buffer C (Tris) was not necessitated, although optional.

Buffer C (Sonication/Lysis, Elution, Dialysis)

50 mM Na-PO₄ buffer pH 6.8 (or substitute with 50 mM Tris phosphate pH 6.8)

100 mM NaCl

10% Glycerol

10 mM β-Mercaptoethanol

0.02% NaN₃

0.25M Sucrose

0.1% Detergent (β-Octyl Glucopyranoside)

1 mM Pefa-Bloc

2 Complete™ tablets (No EDTA)

H₂O to 100 ml.

SDS-Polyacrylamide gel electrophoresis (12.5% acrylamide) and Coommassie Blue staining of the purified protein showed a single band at approximately 70 kD.

The HepC RdRp (NS5B) was assayed by numerous protocols. The simplest method relies on the Novagen Large Scale Transcription Kit (TB069). Modified forms of this protocol have been used successfully. This method is briefly described as follows.

A double stranded DNA template digested upstream of a T7/T3/SP6 promoter is used in the presence of a T7 DNA dependent RNA polymerase to make the RNA template. HepC RdRp (NS5B) in the same cocktail then amplifies the RNA produced by the T7 polymerase.

| | |
|---|---|
| DNA template (0.5 μg/ml)1 μl (0.5 ng) | |
| ATP(20 mM) | 10 μl |
| CTP(20 mM) | 10 μ |
| GTP(20 mM) | 10 μl |
| UTP (20 mM) | 10 μl |
| 5X Transcription buffer | |
| (400 mM HEPES pH7.5, 60 mM MgCl2, 50 mM NaCl) | 20 μl |
| 1M DTT (1M) | 1 μl |
| T7 polymerase (100 U/ml) | 1 μl |
| HepC RdRp (NS5B) | as required |
| Nuclease free water | to 100 μl |

This method has utilized the control DNA template in the kit as well as plasmid DNA cut upstream of the T7 promotor successfully. The quantity of DNA used has been as low as 0.1 ng successfully. The quantity of T7 polymerase used has been as low as 0.1 μl.

Interestingly, the HepC RdRp (NS5B) in these experiments has been demonstrated to possess the capacity to prime off dsDNA in the absence of oligonucleotide primers and amplify RNA.

### Example 14

### Cloning of Hepatitis C RNA Dependent RNA Polymerase Coding Sequence into the Eukaryotic Expression Vector pcDNA3.1

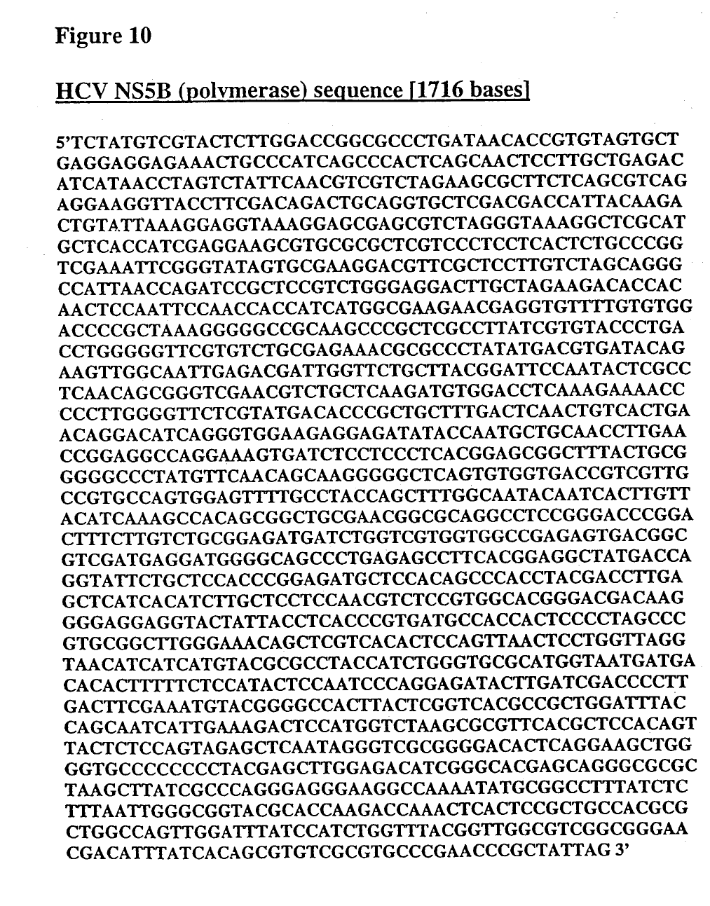

Hepatitis C RNA dependent RNA polymerase coding sequence (FIG. 10) was cloned into the vector pcDNA3.1 (FIG. 9) for expression in situ in the coupled transcription/translation system and concomitant replication/mutation of target RNA. Sequence of oligonucleotides used as primers in PCR amplification of Hepatitis C RNA dependent RNA polymerase for cloning into the EcoRI and NotI restriction sites in the eukaryotic expression vector pcDNA3.1 were:

(SEQ ID NO:33)

5′ GTGGTGGAATTCGCCGCCACCTCTATGTCGTACTCTTGGACC

(SEQ ID NO:34)

5′ GCACGGGCTTGGGCGATAATCCGCCGGCGAGCTCAGATC

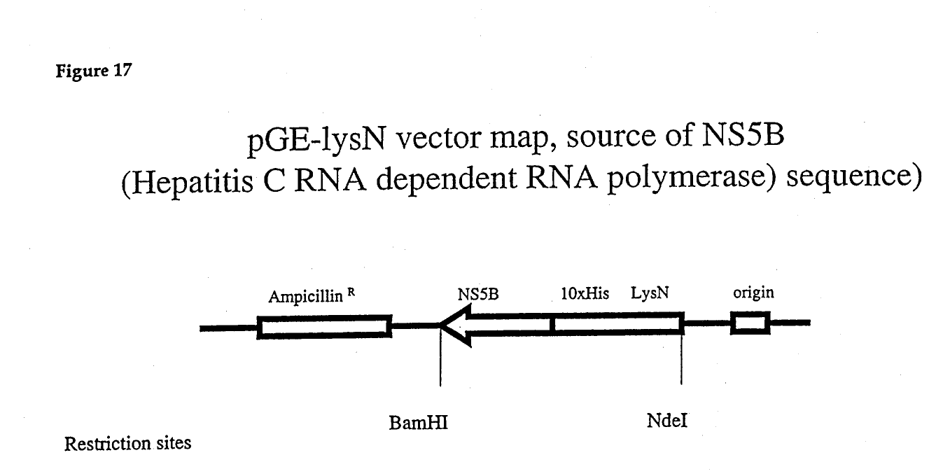

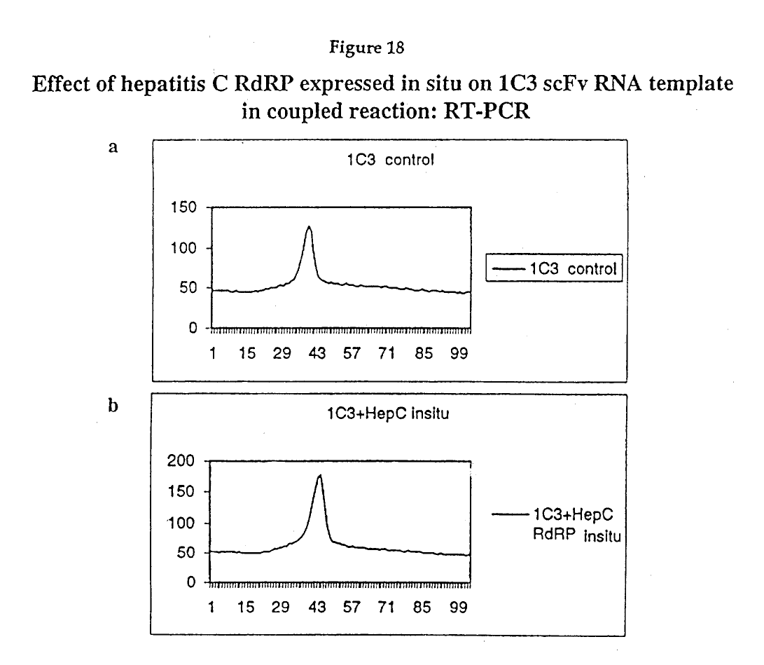

Hepatitis C RNA dependent RNA polymerase was cloned into the pcDNA3.1 vector (named pCDNAHEPC) with a strategy similar to that described in Example 2, but using the above oligonucleotides in the PCR amplification of the Hepatitis C RNA dependent RNA polymerase from the vector shown in FIG. 17. The methods used to demonstrate that the Hepatitis C RNA dependent RNA polymerase were being synthesized in situ were exactly as described in Example 2. The results from the coupled reaction with the Hepatitis C RNA dependent RNA polymerase template in pCDNAHEPC are shown in FIG. 18. The results shown in this figure demonstrate that Hepatitis C RNA dependent RNA polymerase produces larger amounts of transcript (scan b) than T7 polymerase alone. Here, the band has a greater intensity and is broader than the band without Hepatitis C RNA dependent RNA polymerase, indicating the effect on the RNA.

### Example 15

### Use of Qβ Replicase for the Mutation and Selection of β-Lactamase Enzyme With Improved Resistance to Cefotaxime

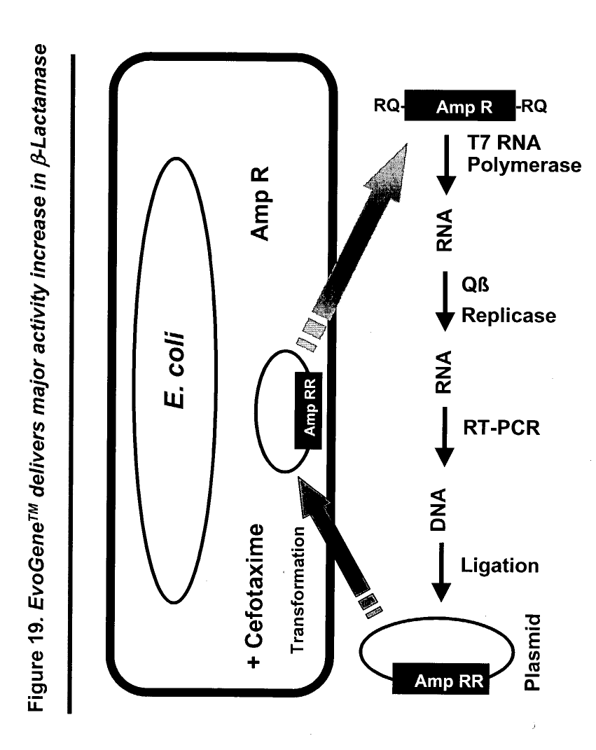

Mutation and selection of β-lactamase with improved resistance to cefotaxime was carried out essentially as depicted in FIG. 19. Briefly, a gene encoding bacterial β-lactamase was ligated into the RQ 135 sequence contained on a DNA vector and transcribed using T7 RNA polymerase. The transcripts were then amplified using Qβ replicase and the conditions outlined below:

RNA template (10 ng)

ATP (200 μM)

CTP (200 μM)

GTP (200 μM)

UTP (200 μM)

Replicase buffer (40 mM Tris-HCl pH 7.9, 21 mM MgCl₂, 10 mM DTT, 2 mM spermidine)

Qβ replicase (1.50 pmol)

Transcripts amplified by Qβ replicase, together with a control population of transcripts that had been processed only with T7 polymerase without exposure to Qβ replicase, were then converted into DNA by RT-PCR. Primers for RT-PCR were:

(SEQ ID NO:37)

5′ CGAGGCGGCCGCGGTCATGAGATTATCAAAAAGG
and

(SEQ ID NO:38)

5′ TCGAGCCATGGCTCATGAGAGACAATAACCCTG.

The reverse transcriptase reaction used standard conditions utilizing SuperScript™ II H-reverse transcriptase (Invitrogen), followed by amplification of the resulting cDNA with Taq polymerase, again, using standard conditions. The resulting DNA molecules were then ligated into a self-replicating prokaryotic plasmid and introduced into E. coli cells by transformation (using standard transformation protocols).

Transformed cells were taken through rounds of enrichment (transformed cells were allowed to grow in rich media for 1 hour at 37° C. prior to being transferred to fresh rich media supplemented with 100 μg/ml ampicillin for 6 hours at 37° C.) and selection (cells were extracted from the ampicillin media and placed into fresh rich media containing either 5 or 20 μg/ml cefotaxime and allowed to grow for 18 hours at 37° C. before being plated onto solid rich media containing either 5 or 20 μg/ml cefotaxime). Several clones resistant to either 5 μg/ml cefotaxime (a 250-fold increase in resistance) or 20 μg/ml cefotaxime (a 1000-fold increase in resistance) were obtained. In contrast, E. coli cells transformed with DNA molecules obtained from the control population of RNA transcripts did not produce any clones resistant to this level of cefotaxime.

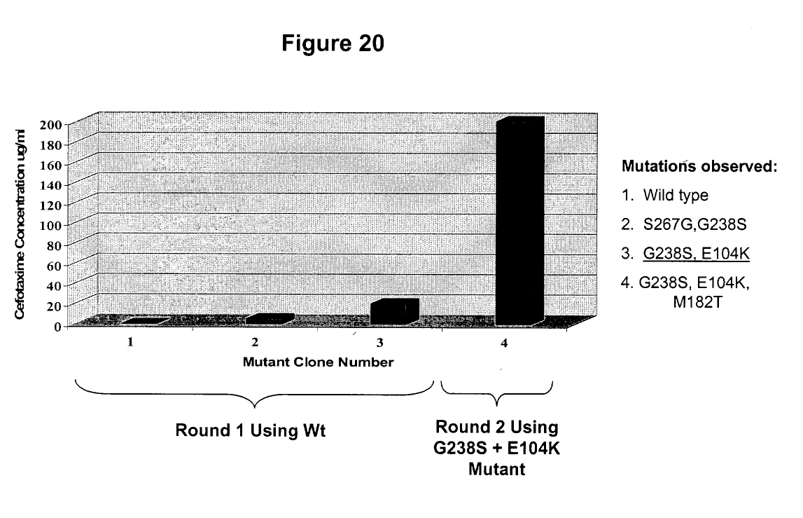

Two of the clones selected after the first round were characterized by sequencing. The genotypes of these mutant clones were (i) S267G, G238S and (ii) G238S, E104K. A second round of mutagenesis and selection was then performed using the mutant clone G238, E104K. Transformed cells were eventually selected with 200 μg/ml cefotaxime. Several clones resistant to 200 μl g/ml cefotaxime were obtained. These clones represent a 10,000-fold increase in cefotaxime resistance. One of these clones was characterized with the genotype G238S, M182T and E104K (see FIG. 20).

Having thus described in detail preferred embodiments of the present invention, it is to be understood that the invention defined by the appended claims is not to be limited to particular details set forth in the above description, as many apparent variations thereof are possible without departing from the spirit or scope of the present invention. Modifications and variations of the method and apparatuses described herein will be obvious to those skilled in the art, and are intended to be encompassed by the following claims.

### REFERENCES

Baranov et al. (1989). Gene 84:463-466.

Baranov et al. (1993). Methods in Enzymology 217:123-142.

Barbas et al. (1994). Ann Rev Imm 57, 191

Barrera et al. (1993). J. Mol. Biol. 232, 512-521.

Brown and Gold (1995a). Biochemistry 34, 14775-14782.

Brown and Gold (1995b). Biochemistry 34, 14765-14774.

Brown and Gold (1996). Proc. Natl. Acad. Sci. (USA) 93, 11558-11562.

Coia et al. (1996). J. Immunol. Meth. 192, 13-23.

Deiman et al (1997), J. Virol. 71, 5990.

Duenas and Borrebaeck (1994). Bio/Technology 12, 999-1002.

Ghee et al. (1996). J. Mol. Biol. 261, 222-230.

Gram et al. (1992). PNAS 89, 3586.

Hanes and Pluckthun (1997). Proc. Natl. Acad. Sci. (USA) 94, 4937-4942.

He and Taussig (1997). Nucleic Acids Research 25, 5132-5134.

Irving et al (1996). Immunotechnology 2, 127-143.

Keiler et al. (1996). Science 271, 990-993.

Kigawa et al. (1991). J. Biochem. 110, 166-168.

Kolosov et al. (1992). Biotechnology and Applied Biochemistry 16, 125-133.

Kramer et al. (1978). Proc. Natl. Acad. Sci. (USA) 75, 5334-5338.

Krebber et al. (1995). FEBS Lett. 377, 227-331.

Kudlicki et al. (1992). Analytical Biochemistry 206, 389-393.

Lizardi et al. (1988). Bio/Technology 6, 1197-1202.

Mattheakis et al. (1994). Proc. Natl. Acad. Sci. (USA) 91, 9022-9026.

Melton et al. (1984a). Nucleic Acid Res. 12: 7035-56.

Melton et al. (1984b). Nucleic Acid Res. 12: 7057-7070.

Morozov et al. (1993). PNAS 90, 9325-9329.

Munishkin et al. (1991). J. Mol. Biol. 221, 463-472.

Nishihara et al. (1983). J. Biochem. (Tokyo) 93, 669-674.

Ojala P M and Bamford D H (1995) Virology, 207, 400.

Rattat et al. (1990). Tibtech 8, 275-276.

Rohde et al. (1995). J Mol Biol 249, 754-762.

Ryabova et al. (1989). Nucleic Acids Research 17, 4412.

Ryabova et al. (1989). Nucleic Acids Research 17, 4412.

Ryabova et al. (1994). Journal of Biological Chemistry 269, 1501-1506.

Ryabova et al. (1997). Nature Biotechnology, 15, 79-84.

Sambrook et al. (1989). In: Nolan, C. (Ed.), Molecular Cloning: A Laboratory Manual, 2nd edn. Cold Spring Harbor Laboratory Press, Cold Spring Harbor, N.Y.

Spirin (1990a) U.S. Pat. Nos. 5,556,769; 5,643,768;

Spirin (1990b). pp. 56-70 in THE RIBOSOME: STRUCTURE, FUNCTION and EVOLUTION (eds. Hill. W. E., et al.), Amer. Soc. Microbiol., Washington, D.C.

Spirin et al. (1988). Science 242, 1162-1164.

Spirin, A. S. (1991). pp 31-43 in FRONTIERS IN BIOPROCESSING II (eds. Todd. P. et al.), Amer. Chem. Soc., Washington, D.C.

Stemmer (1994). Nature 370, 389-391.

Stemmer et al. (1995). Science 270, 1510,

Sumper and Luce (1975). Proc. Natl. Acad. Sci. (USA) 72, 162-166.

Winter et al. (1994). Ann Rev Biochem 12, 433,

Winter et al. (1994). Ann. Rev. Biochem. 12, 433.

Yang et al (1995). J Mol Biol 254, 392

Zamora et al. (1995). Biochemistry, 34, 1261-1266.

Zubay (1973). Ann. Rev. Genetics 7, 267-287

Zucker (1991) Nucleic Acids Res 25; 2707-14

We claim:

1. A method for the mutation, synthesis and selection of a protein of interest, the method comprising:
(a) incubating a replicable RNA molecule encoding the protein with ribonucleoside triphosphate precursors of RNA and an RNA-directed RNA polymerase, wherein the RNA-directed RNA polymerase replicates the RNA molecule but introduces mutations thereby generating a population of mutant RNA molecules;
(b) incubating the mutant RNA molecules with a translation system under conditions which result in the synthesis of a population of mutant proteins wherein, after translation, mutant proteins are linked to their encoding RNA molecules;
(c) selecting one or more mutant proteins of interest.

2. The method as claimed in claim 1, wherein the translation system is a cell-free translation system.

3. The method as claimed in claim 1, wherein the mutant proteins are linked to their encoding RNA molecules via ribosome complexes.

4. The method as claimed in claim 1, wherein the translation system comprises whole cells.

5. The method as claimed in claim 4, wherein the mutant proteins are linked to their encoding RNA molecules by association with or location within the same cell.

6. The method as claimed in claim 1, wherein the selecting in step (c) comprises exposing the mutant protein to a target molecule.

7. The method as claimed in claim 1, wherein the RNA-directed RNA polymerase
(i) introduces mutations into the replicated RNA molecule at a frequency of at least one point mutation in 10⁴ bases; or
(ii) introduces at least one insertion or deletion at a frequency of 10⁻⁴.

8. The method as claimed in claim 1, wherein the RNA-directed RNA polymerase
(i) introduces mutations into the replicated RNA molecule at a frequency of at least one point mutation in 10³ bases; or
(ii) introduces at least one insertion or deletion at a frequency of 10⁻³.

9. The method as claimed in claim 1, wherein the RNA-directed RNA polymerase is selected from the group consisting of Qβ replicase, Hepatitis C RNA-directed RNA polymerase, Vesicular Stomatitis Virus RNA-directed RNA polymerase, Turnip yellow mosaic virus replicase and RNA bacteriophage phi 6 RNA-dependent RNA polymerase.

10. The method as claimed in claim 1, wherein the RNA-directed RNA polymerase is Qβ replicase.

11. A method for producing and selecting a mutant protein of interest, the method comprising:
(a) incubating a replicable RNA molecule encoding the protein with ribonucleoside triphosphate precursors of RNA and an RNA-directed RNA polymerase, wherein the RNA-directed RNA polymerase replicates the RNA molecule but introduces mutations thereby generating a population of mutant RNA molecules;
(b) translating the mutant RNA molecules in cells wherein, after translation, each mutant protein is associated with or located within the same cell as its encoding RNA molecule; and
(c) selecting a cell comprising a mutant protein of interest.

12. The method as claimed in claim 11, wherein the selecting in step (c) comprises exposing the cells to a target molecule.

13. The method as claimed in claim 11, which further comprises the step of recovering the mutant RNA molecule or the corresponding DNA molecule encoding the mutant protein of interest from the cell selected in step (c).

14. The method as claimed in claim 13, which further comprises repeating steps (a) to (c).

15. The method as claimed in claim 11, wherein the RNA-directed RNA polymerase
(i) introduces mutations into the replicated RNA molecule at a frequency of at least one point mutation in 10⁴ bases; or
(ii) introduces at least one insertion or deletion at a frequency of 10⁻⁴.

16. The method as claimed in claim 11, wherein the RNA-directed RNA polymerase
(i) introduces mutations into the replicated RNA molecule at a frequency of at least one point mutation in 10³ bases; or
(ii) introduces at least one insertion or deletion at a frequency of 10⁻³.

17. The method as claimed in claim 11, wherein the RNA-directed RNA polymerase is selected from the group consisting of Qβ replicase, Hepatitis C RNA-directed RNA polymerase, Vesicular Stomatitis Virus RNA-directed RNA polymerase, Turnip yellow mosaic virus replicase and RNA bacteriophage phi 6 RNA-dependent RNA polymerase.

18. The method as claimed in claim 11, wherein the RNA-directed RNA polymerase is Qβ replicase.

19. A method for producing and selecting a mutant protein of interest, the method comprising:
(a) transcribing a DNA template to produce a replicable RNA molecule, wherein the DNA template comprises:
(i) an untranslated region comprising a control element that promotes transcription of DNA into RNA and a ribosome binding site;
(ii) an open reading frame encoding a protein; and
(iii) a stemloop structure situated upstream of the open reading frame;
(b) incubating the replicable RNA molecule encoding the protein with ribonucleoside triphosphate precursors of RNA and an RNA-directed RNA polymerase, wherein the RNA-directed RNA polymerase replicates the RNA molecule but introduces mutations, thereby generating a population of mutant RNA molecules;
(c) incubating the mutant RNA molecules with a translation system under conditions which result in the synthesis of a population of mutant proteins;
(d) selecting one or more mutant proteins of interest.

20. The method as claimed in claim 19, wherein the translation system comprises intact cells.

21. The method as claimed in claim 19, wherein the selecting in step (d) comprises exposing the cells to a target molecule.


SEQUENCE LISTING (38 sequences)

SEQ ID NO: 1 - DNA, 318 bp, Murine sp.
  misc_feature (1)..(318): Sequence of the constant light region of mouse monoclonal antibody 1C
gctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggggagagtgt

SEQ ID NO: 2 - DNA, 324 bp, Homo sapiens
  misc_feature (1)..(324): Sequence of the human constant heavy chain
gcagatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggc

SEQ ID NO: 3 - DNA, 748 bp, Homo Sapeins
  misc_feature (1)..(748): Sequence of the anti-glycophorin (1C3) scFv
atggccgaggtgaggcttcttgagtctggaggtggcccggtacaacctggaggatccctgaaactctcctgtgcagcctcaggattcgattttagtagatactggatgaattgggtccggcgggctccagggaaggggctagagtggattggagaaattaatcaacaaagcagtacgataaactattcgccacctctgaaggataaattcatcatctccagagacaacgccaaaagtacgctgtacctgcaaatgaacaaagtgagatctgaggacacagccctttattattgtgcaagactttctcttactgcggcagggtttgcttactggggccaagggactctggtcaccgtcgcctccggtggtggtggttcaggaggaggaggttcgggtggtggtggttcggacatcgtcatgtcacagtctccatcctccctggctgtgtcagtaggagagaaggtcactatgagctgcagatccagtcagagtctgttcaacagtagaacccgaaagaactacttgacttggtaccagcagaaaccagggcagtctcctaaaccgctgatctactgggcatccactagggaatctggggtccctgatcgcttcacaggcagtggatctgggacagatttcactctcaccatcagcagtgtgcaggctgaagacctggcagattattactgcaagcaatcttataatcttcggacgttcggtggaggcaccaagctggaaatcaaacggg

SEQ ID NO: 4 - DNA, 807 bp, Homo Sapeins
  misc_feature (1)..(807): Sequence of the anti-hepatitis surface antigen (4C2) scFv
ccatggccgatgtgaagcttcaggagtcagggcctgagctggtgaggcccggggtctcagtgaagattacctgcaagggttccggctacacattcactgattatgctatgcattgggtgaagcagagtcatgccaagagtctagagtggattggacttattagtaattcctttggtaatacaaactacaaccagaagtttgaggccaaggccacaatgactgtagacaaatcctccaacacaggctatttggaacttggcagattgacatctgaggattctgccatctattactgtgcaagagtgatcgactggtccttcgatgtctggggccaagggaccacggtcaccgtctcctcaggtggaggcggttcaggcggaggtggctctggcggtggcggatcggacattgtgctgacccaatctccagcaatcatgttcgcatctccaggggagaaggtcaccatgacctgcagtgccaactcacgtgtcaggtacgtgcactggtaccaacagaagtcaggcacctcccccaaaagatggatttatgacacatccaaactggcttctggagtccctgctcgcttcagtggcagtgggtctgggacctctcactctctcacaatcagcagcttggaggctgaagatgctgccacttattactgccagcactggagtagtaaccctcccacgttcggtgctgggaccaagctggaaataaaacgggcggccgcagattataaagatgatgatgataaagccgcggcccatcaccaccatcaccattaagaattcagcccgcctaatg

SEQ ID NO: 5 - DNA, 7489 bp, QB Bacteriophage
ggggaccccctttagggggtcacctcacacagcagtacttcactgagtataagaggacatatgcctaaattaccgcgtggtctgcgtttcggagccgataatgaaattcttaatgattttcaggagctctggtttccagacctctttatcgaatcttccgacacgcatccgtggtacacactgaagggtcgtgtgttgaacgcccaccttgatgatcgtctacctaatgtaggcggtcgccaggtaaggcgcactccacatcgcgtcaccgttccgattgcctcttcaggccttcgtccggtaacaaccgttcagtatgatcccgcagcactatcgttcttattgaacgctcgtgttgactgggatttcggtaatggcgatagtgcgaaccttgtcattaatgactttctgtttcgcacctttgcacctaaggagtttgatttttcgaactccttagttcctcgttatactcaggccttctccgcgtttaatgccaagtatggcactatgatcggcgaagggctcgagactataaaatatctcgggcttttactgcgcagactgcgtgagggttaccgcgctgttaagcgtggcgatttacgtgctcttcgtagggttatccagtcctaccataatggtaagtggaaaccggctactgctggtaatctctggcttgaatttcgttatggccttatgcctctcttttatgacatcagagatgtcatgttagactggcagaaccgtcatgataagattcaacgcctccttcggttttctgttggtcacggcgaggattacgttgtcgaattcgacaatctgtaccctgccgttgcttactttaaactgaaaggggagattacactcgaacgccgtcatcgtcatggcatatcttacgctaaccgcgaaggatatgctgttttcgacaacggttcccttcggcctgtgtccgattggaaggagcttgccactgcattcatcaatccgcatgaagttgcttgggagttaactccctacagcttcgttgttgattggttcttgaatgttggtgacatacttgctcaacaaggtcagctatatcataatatcgatattgtagacggctttgacagacgtgacatccggctcaaatctttcaccataaaaggtgaacgaaatgggcggcctgttaacgtttctgctagcctgtctgctgtcgatttattttacagccgactccatacgagcaatcttccgttcgctacactagatcttgatactacctttagttcgtttaaacacgttcttgatagtatctttttattaacccaacgcgtaaagcgttgaaactttgggtcaatttgatcatggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattgaacactgctcattgccggtggtggctcagggtcaaaacccgatccggttattccggatccaccgattgatccgccgccagggacaggtaagtatacctgtcccttcgcaatttggtccctagaggaggtttacgagcctcctactaagaaccgaccgtggcctatctataatgctgttgaactccagcctcgcgaatttgatgttgccctcaaagatcttttgggcaatacaaagtggcgtgattgggattctcggcttagttataccacgttccgcggttgccgtggcaatggttatattgaccttgatgcgacttatcttgctactgatcaggctatgcgtgatcagaagtatgatattcgcgagggcaagaaacctggtgctttcggtaacattgagcgattcatttatcttaagtcgataaatgcttattgctctcttagcgatattgcggcctatcacgccgatggcgtgatagttggcttttggcgcgatccatccagtggtggtgccataccgtttgacttcactaagtttgataagactaaatgtcctattcaagccgtgatagtcgttcctcgtgcttagtaactaaggatgaaatgcatgtctaagacagcatcttcgcgtaactctctcagcgcacaattgcgccgagccgcgaacacaagaattgaggttgaaggtaacctcgcactttccattgccaacgatttactgttggcctatggtcagtcgccatttaactctgaggctgagtgtatttcattcagcccgagattcgacgggaccccggatgactttaggataaattatcttaaagccgagatcatgtcgaagtatgacgacttcagcctaggtattgataccgaagctgttgcctgggagaagttcctggcagcagaggctgaatgtgctttaacgaacgctcgtctctataggcctgactacagtgaggatttcaatttctcactgggcgagtcatgtatacacatggctcgtagaaaaatagccaagctaataggagatgttccgtccgttgagggtatgttgcgtcactgccgattttctggcggtgctacaacaacgaataaccgttcgtacggtcatccgtccttcaagtttgcgcttccgcaagcgtgtacgcctcgggctttgaagtatgttttagctctcagagcttctacacatttcgatatcagaatttctgatattagcccttttaataaagcagttactgtacctaagaacagtaagacagatcgttgtattgctatcgaacctggttggaatatgtttttccaactgggtatcggtggcattctacgcgatcggttgcgttgctggggtatcgatctgaatgatcagacgataaatcagcgccgcgctcacgaaggctccgttactaataacttagcaacggttgatctctcagcggcaagcgattctatatctcttgccctctgtgagctcttattgcccccaggctggtttgaggttcttatggacctcagatcacctaaggggcgattgcctgacggtagtgttgttacctacgagaagatttcttctatgggtaacggttacacattcgagctcgagtcgcttatttttgcttctctcgctcgttccgtttgtgagatactggacttagactcgtctgaggtcactgtttacggagacgatattattttaccgtcctgtgcagtccctgccctccgggaagtttttaagtatgttggttttacgaccaatactaaaaagactttttccgaggggccgttcagagagtcgtgcggcaagcactactattctggcgtagatgttactcccttttacatacgtcaccgtatagtgagtcctgccgatttaatactggttttgaataacctatatcggtgggccacaattgacggcgtatgggatcctagggcccattctgtgtacctcaagtatcgtaagttgctgcctaaacagctgcaacgtaatactatacctgatggttacggtgatggtgccctcgtcggatcggtcctaatcaatcctttcgcgaaaaaccgcgggtggatccggtacgtaccggtgattacggaccatacaagggaccgagagcgcgctgagttggggtcgtatctctacgacctcttctcgcgttgtctctcggaaagtaacgatgggttgcctcttaggggtccatcgggttgcgattctgcggatctatttgccatcgatcagcttatctgtaggagtaatcctacgaagataagcaggtctaccggcaaattcgatatacagtatatcgcgtgcagtagccgtgttctggcaccctacggggtcttccagggcacgaaggttgcgtctctacacgaggcgtaacctgggagggcgccaatatggcgcctaattgtgaataaattatcacaattactcttacgagtgagagggggatctgctttgccctctctcctcccgggggatccactagttctaggtactcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctacgcaactgttgggaagggcgatcggtgcgggcctcttcgctattacgccagctggcgaaggggggatgtgctgcaaggcgattaagttgggtaacgccagggttttcccagtcacgacgttgtaaaacgacggccagtgaattgtaatacgactcactata

SEQ ID NO: 6 - DNA, 1716 bp, hepatitis C virus
  misc_feature (1)..(176): DNA sequence of the hepatitis C virus RNA dependent RNA polymerase
tctatgtcgtactcttggaccggcgccctgataacaccgtgtagtgctgaggaggagaaactgcccatcagcccactcagcaactccttgctgagacatcataacctagtctattcaacgtcgtctagaagcgcttctcagcgtcagaggaaggttaccttcgacagactgcaggtgctcgacgaccattacaagactgtattaaaggaggtaaaggagcgagcgtctagggtaaaggctcgcatgctcaccatcgaggaagcgtgcgcgctcgtccctcctcactctgcccggtcgaaattcgggtatagtgcgaaggacgttcgctccttgtctagcagggccattaaccagatccgctccgtctgggaggacttgctagaagacaccacaactccaattccaaccaccatcatggcgaagaacgaggtgttttgtgtggaccccgctaaagggggccgcaagcccgctcgccttatcgtgtaccctgacctgggggttcgtgtctgcgagaaacgcgccctatatgacgtgatacagaagttggcaattgagacgattggttctgcttacggattccaatactcgcctcaacagcgggtcgaacgtctgctcaagatgtggacctcaaagaaaacccccttggggttctcgtatgacacccgctgctttgactcaactgtcactgaacaggacatcagggtggaagaggagatataccaatgctgcaaccttgaaccggaggccaggaaagtgatctcctccctcacggagcggctttactgcgggggccctatgttcaacagcaagggggctcagtgtggtgaccgtcgttgccgtgccagtggagttttgcctaccagctttggcaatacaatcacttgttacatcaaagccacagcggctgcgaacggcgcaggcctccgggacccggactttcttgtctgcggagatgatctggtcgtggtggccgagagtgacggcgtcgatgaggatggggcagccctgagagccttcacggaggctatgaccaggtattctgctccacccggagatgctccacagcccacctacgaccttgagctcatcacatcttgctcctccaacgtctccgtggcacgggacgacaaggggaggaggtactattacctcacccgtgatgccaccactcccctagcccgtgcggcttgggaaacagctcgtcacactccagttaactcctggttaggtaacatcatcatgtacgcgcctaccatctgggtgcgcatggtaatgatgacacactttttctccatactccaatcccaggagatacttgatcgaccccttgacttcgaaatgtacggggccacttactcggtcacgccgctggatttaccagcaatcattgaaagactccatggtctaagcgcgttcacgctccacagttactctccagtagagctcaatagggtcgcggggacactcaggaagctggggtgcccccccctacgagcttggagacatcgggcacgagcagggcgcgctaagcttatcgcccagggagggaaggccaaaatatgcggcctttatctctttaattgggcggtacgcaccaagaccaaactcactccgctgccacgcgctggccagttggatttatccatctggtttacggttggcgtcggcgggaacgacatttatcacagcgtgtcgcgtgcccgaacccgctattag

SEQ ID NO: 7 - DNA, 65 bp, QB Bacteriophage
gcgcgaatacgactcactatagagggacaaaccgccatggccgaggtgaggcttcttgagtctgg

SEQ ID NO: 8 - DNA, 58 bp, QB BActeriophage
catcatcatcatctttataatctgcggccgcacactctcccctgttgaagctcttgac

SEQ ID NO: 9 - DNA, 57 bp, QB Bacteriophage
cccctgttgaagctcttgacaatgggtgaagttgatgtcttgtgagtggcctcacag

SEQ ID NO: 10 - DNA, 46 bp, QB Bacteriophage
cttgtgagtggcctcacaggtatagctgttatgtcgttcatactcg

SEQ ID NO: 11 - DNA, 55 bp, QB Bacteriophage
accatgattacgccaagctctaatacgactcactatagggaaagctcgcttgttc

SEQ ID NO: 12 - DNA, 60 bp, QB Bacteriophage
agggaaagctcgcttgttctttttgcagaagctcagaataaacgctcaactttggccacc

SEQ ID NO: 13 - DNA, 39 bp, QB Bacteriophage
tttataatctgcggccgccgcctcgtgtagagacgcaac

SEQ ID NO: 14 - DNA, 48 bp, QB Bacteriophage
ttactcgcggcccagccggccatggccatgtctaagacagcatcttcg

SEQ ID NO: 15 - DNA, 60 bp, QB Bacteriophage
gcagctaatacgactcactataggaacagaccaccatggacgtggcccagcctgctgtgg

SEQ ID NO: 16 - DNA, 50 bp, QB Bacteriophage
aaacgctcaactttggccaccatggatgtgaagcttcaggagtctgggcc

SEQ ID NO: 17 - DNA, 51 bp, QB Bacteriophage
gcccttgggccgggagatggtctgcttcagtggcgagggcaggtctgtgtg

SEQ ID NO: 18 - DNA, 49 bp, QB Bacteriophage
cgagggcaggtctgtgtgggtcacggtgcacgtgaacctctccccggag

SEQ ID NO: 19 - DNA, 51 bp, QB Bacteriophage
cgtgaacctctccccggagttccagtcatcctcgcagatgctggcctcacc

SEQ ID NO: 20 - DNA, 65 bp, QB Bacteriophage
gcgcgaatacgactcactatagagggacaaaccgccatggccgatgtgaagcttcaggagtcagg

SEQ ID NO: 21 - DNA, 60 bp, QB Bacteriophage; identical to SEQ ID NO: 15

SEQ ID NO: 22 - DNA, 54 bp, QB Bacteriophage
taatacgactcactatagggaaagggtttctccgatccgggaacataggatacc

SEQ ID NO: 23 - DNA, 61 bp, QB Bacteriophage
tgaggtatcctatgttcccggatcggagaaacccacactctcccctgttgaagctcttgac

SEQ ID NO: 24 - DNA, 56 bp, QB Bacteriophage
ccgggaacataggatacctcaaccaccatggccgaggtgaggcttcttgagtctgg

SEQ ID NO: 25 - RNA, 28 nt, Unknown
  sequence of the stemloop structure for binding the QB replicase
gggacacgaaagccccaggaaccuuucg

SEQ ID NO: 26 - DNA, 23 bp, Bacteriophage T7
taatacgactcactatagggaga

SEQ ID NO: 27 - protein, 8 aa, Bacteriophage T7
DYKDDDDK

SEQ ID NO: 28 - DNA, 42 bp, QB Bacteriophage
tctgcagaattcgccgccaccatgtctaagacagcatcttcg

SEQ ID NO: 29 - DNA, 39 bp, QB Bacteriophage
tttataarctgcggccgcttacgcctcgtgtagagacgc

SEQ ID NO: 30 - DNA, 42 bp, bacteriophage T7
gcgcgaatacgactcactatagagggacaaaccgccatggcc

SEQ ID NO: 31 - DNA, 41 bp, bacteriophage T7
gcagctaatacgactcactataggaacagaccaccatggcc

SEQ ID NO: 32 - protein, 20 aa, Artificial
  linker between LysN and NS5B
GSGSGHHHHHHHHHHDDDDK

SEQ ID NO: 33 - DNA, 42 bp, Homo Sapien
gtggtggaattcgccgccacctctatgtcgtactcttggacc

SEQ ID NO: 34 - DNA, 39 bp, Homo Sapien
gcacgggcttgggcgataatccgccggcgagctcagatc

SEQ ID NO: 35 - RNA, 54 nt, Unknown
  sequence of RNA fragment that forms stemloop structure
gggguuuccgggaacauaggauaccucaucucuauagugagucguauuuuccca

SEQ ID NO: 36 - RNA, 57 nt, Unknown
  sequence of RNA fragment that forms stemloop structure
gcgcgaauacgacucaucauagagggacaaaccaugaggucaaacucgagagucagg

SEQ ID NO: 37 - DNA, 34 bp, bacteriophage T7
cgaggcggccgcggtcatgagattatcaaaaagg

SEQ ID NO: 38 - DNA, 33 bp, bacteriophage T7
tcgagccatggctcatgagagacaataaccctg